(19) United States
(12) Patent Application Publication
(10) Pub. No.: US 2012/0142722 A1
(43) Pub. Date: Jun. 7, 2012

(54) CONJUGATES OF CYTOTOXIC DRUGS

(76) Inventors: Ilan Ziv, Kfar Saba (IL); Anat Shirvan, Herzliya (IL); Hagit Grimberg, Herzliya (IL)

(21) Appl. No.: 13/380,120

(22) PCT Filed: Jun. 23, 2010
(86) PCT No.: PCT/IL10/00499

§ 371 Date: Feb. 19, 2012

Related U.S. Application Data
(60) Provisional application No. 61/220,337, filed on Jun. 25, 2009.

(51) Int. Cl. A61K 31/4745; A61K 31/196; C07C 275/68; C07D 498/22; A61P 35/00

(52) U.S. Cl. 514/283; 546/48; 562/507; 514/565

(57) ABSTRACT

Novel compounds and pharmaceutical compositions are provided. In one aspect of the invention the compounds may be utilized in medical practice, for example, in treatment of cancer and immune disorders. In another aspect of the invention there is provided a conjugate, comprising a cytotoxic agent and a modulating moiety, the modulating moiety serving to target apoptotic cells.

### FIELD OF THE INVENTION

The invention relates to the field of medical disorders and their treatment. Specifically, the invention relates to novel compounds and pharmaceutical compositions. The compounds may be utilized in medical practice, for example, in treatment of cancer and immune disorders.

### BACKGROUND OF THE INVENTION

Cancer is a prevalent medical disorder and a leading cause of death, which continuously calls for novel therapeutic strategies.

Known anti-cancer agents include, among others, alkylating agents and topoisomerase inhibitors.

Alkylating agents attach an alkyl group to DNA and other macromolecules. Some examples of alkylating agents which are used clinically to treat a variety of tumors include Lomustine (also known as Chloroethylcyclo-hexylnitrosourea (CCNU)) and Carmustine (also known as 1,3-Bis(2-chloroethyl)-1-nitrosourea (BCNU)). These drugs are used, among others, for treatment of brain tumors. Topoisomerase inhibitors are involved in inhibiting topoisomerases which are enzymes essential to maintaining the topology of DNA. Inhibition of these enzymes interferes with both transcription and replication of the DNA by upsetting proper DNA supercoiling. Type I topoisomerase inhibitors include, for example, camptothecines: Irinotecan and topotecan.

Cancer cells are typically more sensitive to induction of DNA damage by anti-cancer agents than healthy cells since cancer cells generally proliferate more than healthy cells. However, anti-cancer agents, which are usually inherently cytotoxic, typically cause side effects in healthy tissue, particularly in healthy tissue where cell proliferation is frequent, such as in the gastrointestinal tract or bone marrow.

Thus, even though there are numerous agents with proven cytotoxic activity, effective tumor control is often limited by concomitant induction of adverse effects, due to damage to healthy tissue. Common side effects include nausea, vomiting and diarrhea, due to gastrointestinal toxicity, and a decrease in white and red blood cells and platelets. This exposes the patient to an increased risk of infection, anemia and bleeding.

Therefore, researchers are continuously seeking drugs with better efficacy and improved drug performance in parameters such as oral bioavailability, selectivity and an improved pharmacokinetic profile.

Pro-drugs are sometimes used to advance parameters such as oral bioavailability, selectivity and pharmacokinetics of drugs. A pro-drug is a pharmacological substance in a typically inactive form, which upon administration is metabolized in vivo into an active metabolite. An anti-cancer agent designed as a pro-drug may have low cytotoxicity prior to its activation, thus lowering the chance of its “attacking” healthy, non-cancerous cells.

Although some solutions for improved parameters and reduced side effects of drugs exist, improvement of the efficacy/toxicity balance of cytotoxic drugs, for example in anticancer or immune disorder treatments, is still highly needed.

### SUMMARY OF THE INVENTION

Embodiments of the invention provide novel compounds and novel pharmaceutical compositions.

According to further embodiments, compounds, pharmaceutical compositions and methods for their use are provided to achieve an effective profile of tumor exposure to a cytotoxic drug compared to that provided by the prior art. The novel compounds and pharmaceutical compositions of the invention may be used to achieve effective performance of cytotoxic drugs in vivo.

According to yet further embodiments of the invention, there is provided a conjugate, comprising a cytotoxic agent and a modulating moiety which may modulate the performance of the cytotoxic drug upon administration. Optionally, the cytotoxic agent may be attached to the modulating moiety through a linker.

The term “conjugate” in the present invention relates to a molecule produced by chemically uniting two (or more) compounds (e.g., a drug and a modulating moiety).

The term “modulating moiety” in the present invention relates to a chemical constituent attached to a molecule, preferably to a drug, typically for modulating the performance of the drug, such as its distribution pattern upon administration, its metabolism or its excretion. Preferably, the moiety enhances the efficacy/toxicity balance of a cytotoxic drug (for example an anticancer agent). In addition, the modulating moiety may confer other advantages to the conjugate over an un-conjugated cytotoxic agent, for example, stability (e.g., by adhering to stabilizing proteins in vivo), or an improved pharmacokinetic profile.

The term “therapeutically-effective amount” relates to an amount of drug that elicits a therapeutically useful response, in treatment of an existing medical disorder, and/or in preventing or delaying the disease onset, in an animal or a human subject.

The term “cytotoxic agent” relates to a chemical compound used for eradication of tumor cells.

A conjugate according to embodiments of the invention may be advantageous over the cytotoxic agent alone, in one or more aspects of performance, such as, for example, efficacy, toxicity, or pharmacokinetics. According to one embodiment of the invention, the conjugate may act as a pro-drug, in that while the conjugate is intact the drug or cytotoxic agent is inactive, whereas upon administration and metabolism in the body, an active element is released, to exert its effect on its target sites.

Compounds according to embodiments of the invention may be described by the general formula (I):

Z-L-D (Formula I)

wherein D is a drug, such as a cytotoxic agent; L is either null (absent) or a linker, being either stable or cleavable in physiological conditions; and Z is a modulating moiety, as defined above.

According to some embodiments D is a drug, typically a cytotoxic agent, used for the treatment of cancer and/or for modulation of the immune system.

According to some embodiments, the modulating moiety targets the compounds to tumor cells. According to additional embodiments the modulating moiety targets the compounds to cells undergoing cell death (e.g., by apoptosis), for example, within a tumor or at a site of an immune disorder.

According to one embodiment there is provided a compound represented by the structure as set forth in Formula III

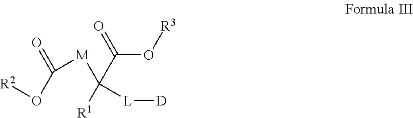

In which R¹, R², R³ are each independently selected from hydrogen, or C₁, C₂, C₃, C₄, C₅, C₆, C₇, C₈ linear or branched alkyl, aryl or heteroaryl, which may contain one or two rings; M is either absent or is a C₁, C₂, C₃, or C₄ alkylene; D is a cytotoxic agent; and L is either absent or is a C₁, C₂, C₃, C₄, C₅, C₆, C₇, C₈ linear or branched, substituted or unsubstituted, alkylene, aryl or heteroaryl, which may contain one or two rings, carbamate, amide or ester or combinations thereof.

According to one embodiment D is camptothecin, camptothecin analogue or a nitrosurea.

According to some embodiments R¹, R² and R³ are each independently selected from hydrogen, C₁, C₂, C₃, C₄, C₅, C₆ linear or branched alkyl; D is camptothecin, camptothecin analogue or nitrosurea; M is absent; and L is either absent or is a C₁, C₂, C₃, C₄, C₅, C₆, C₇, C₈ linear or branched, substituted or unsubstituted, alkylene, aryl or heteroaryl, which may contain one or two rings, carbamate, amide or ester or combinations thereof.

According to one embodiment of the invention there is provided a compound represented by the structure as set forth in Formula II

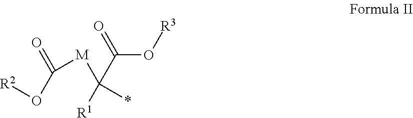

In which R¹, R², R³ are each independently selected from hydrogen, or C₁, C₂, C₃, C₄, C₅, C₆, C₇, C₈ linear or branched alkyl, aryl or heteroaryl, which may contain one or two rings, or combinations thereof; M is either absent or a C₁, C₂, C₃, or C₄ alkylene; and * indicates a linkage point, either directly or through a linker to a cytotoxic agent.

The cytotoxic agent may be camptothecin, camptothecin analogue, or nitrosurea.

According to one embodiment M is absent. According to one embodiment R¹ is methyl.

According to some embodiments R² and R³ are each independently selected from hydrogen, C₁, C₂, C₃, C₄, C₅, C₆ branched or linear alkyl.

According to some embodiments the compound represented by Formula II comprises a linker connected to the linkage point, said linker selected from C₁, C₂, C₃, C₄, C₅, C₆, C₇, C₈ linear or branched, optionally substituted, alkylene, aryl or heteroaryl, composed of one or two rings, carbamate, amide or ester groups or combinations thereof.

According to one embodiment there is provided a pharmaceutical composition comprising the compound according to the embodiments of the invention and a pharmaceutically acceptable salt or carrier.

According to another embodiment there is provided a method for the treatment of cancer and/or immune disorders, the method comprising administering to a subject a therapeutically effective amount of the pharmaceutical composition.

### BRIEF DESCRIPTION OF THE FIGURES

The invention will now be described in relation to certain examples and embodiments with reference to the following illustrative figures so that it may be more fully understood. In the drawings:

FIG. 1 is a diagram showing the efficacy of ATT-1 (a conjugate according to one embodiment of the invention), as a cytotoxic anti-cancer drug, as exemplified in cultured A375-melanoma cells in vitro. A375 cells were incubated with increasing concentrations of ATT-1 and cell viability was assessed. The cytotoxic effect of ATT-1 was expressed as the percentage of viable cells at 48 hours of incubation. ATT-1 exhibited potent cytotoxic effect, with IC₅₀ (inhibitory concentration of 50% of cells) of 545 μM, showing ATT-1 to be a potent anti-cancer agent;

FIG. 2 is a diagram showing the performance of ATT-1 in inhibiting tumor growth in tumor-bearing nude mice. A375 melanoma tumor-bearing mice were treated with a first dose of ATT-1 at 50 mg/kg on day 8 (post tumor cell inoculation), and a second dose of 40 mg/kg on day 15. Tumor volumes (mm³) were measured, in comparison with the tumor volumes in control, untreated mice. ATT-1 exerted an inhibitory effect on the tumors, with tumor regression and growth inhibition of 73% compared with the control;

FIG. 3 is a diagram showing the performance of ATT-1 in inhibiting tumor growth in tumor-bearing nude mice, compared with an equal total dose of CCNU. A375 melanoma tumor-bearing mice were treated with equal amounts (mg/kg) of ATT-1 vs. CCNU. Tumor volumes (mm³) were measured, in comparison with the tumor volumes in control, untreated mice. ATT-1 exerted a slightly greater inhibitory effect on the tumors than CCNU, showing ATT-1 to be an effective tumor growth inhibitor;

FIG. 4 is a diagram showing the performance of ATT-1 in tumor-bearing nude mice, compared with equi-molar dose of CCNU in inhibiting tumor growth. A375 melanoma tumor-bearing mice were treated with equi-molar amounts of ATT-1 vs. CCNU. Tumor volumes (mm³) were measured, in comparison with the tumor volumes in control, untreated mice. ATT-1 showed 76% growth inhibition vs. 48% growth inhibition induced by CCNU;

FIG. 5 is a diagram showing no substantial adverse effect of ATT-1 on body weight in the melanoma A375 tumor-bearing mice as compared with CCNU. Tumor-bearing mice which were treated with ATT-1 and CCNU were weighed twice a week. ATT-1 showed less effect on animal body weight than CCNU;

FIG. 6 is a diagram showing the effect of repeated doses of ATT-1 and CCNU on white blood cell counts in A375 tumor bearing mice. Equal total dose of ATT-1 and CCNU were administered to the mice and white blood cell count (WBC) was tested after the last dose. A markedly lower affect on WBC was demonstrated for ATT-1 treated animals than for CCNU treated animals indicating that ATT-1 treatment preserves white blood cell count and does not cause a reduction of white blood cells;

FIG. 7 is a diagram showing the efficacy of ATT-5E (a conjugate according to another embodiment of the invention), as a cytotoxic anti-cancer drug, as exemplified in cultured A375-melanoma cells in vitro. A375 cells were incubated with increasing concentrations of ATT-5E and CCNU and cell viability was assessed. The cytotoxic effects of ATT-5E and of CCNU were expressed as the percentage of viable cells at 48 hours of incubation. ATT-5E exhibited potent cytotoxic effect, with IC₅₀ (inhibitory concentration of 50% of cells) of 130 μM whereas CCNU exhibited an IC₅₀ of 200 μM. These results indicate a higher cell killing effect of ATT-5E compared to CCNU.

FIG. 8 is a diagram showing the efficacy of ATT-11E (a conjugate according to another embodiment of the invention), as a cytotoxic anti-cancer drug, as exemplified in cultured A375-melanoma cells in vitro. A375 cells were incubated with increasing concentrations of ATT-11E and Irinotecan (CPT-11) and cell viability was assessed. The cytotoxic effects of ATT-11E and Irinotecan were expressed as the percentage of viable cells at 48 hours of incubation. ATT-11E exhibited potent cytotoxic effect, with IC₅₀ (inhibitory concentration of 50% of cells) of 0.6 μM whereas Irinotecan exhibited an IC₅₀ of 44 μM. Thus, a clear cell killing effect of ATT-11E and a dramatically higher cytotoxicity of ATT-11E compared to Irinotecan was shown in vitro;

FIG. 9 is a diagram showing the performance of ATT-11E in inhibiting tumor growth in tumor-bearing nude mice. A375 melanoma tumor-bearing mice were treated with ATT-11E in two different dosing regimens. Tumor volumes (mm³) were measured, in comparison with the tumor volumes in control, untreated mice. In both regimens a dramatic inhibition of tumor growth was exhibited (97% tumor volume growth inhibition in regimen 1 and 90% inhibition in regimen II), showing ATT-11E to be a potent agent for tumor growth inhibition by two different dose regimens;

FIGS. 10A-B show the performance of ATT-11E in inhibiting tumor growth in tumor-bearing nude mice in a dose response manner. A375 melanoma tumor-bearing mice were treated with ATT-11E in different dosing regimens. Tumor volumes (mm³) were measured, in comparison with the tumor volumes in control, untreated mice. ATT-11E was found to be a potent tumor growth inhibiting agent, showing a clear dose response;

FIG. 11 demonstrates the anti-cancer effect of ATT-11E, causing regression and growth inhibition of melanoma A375 tumors in nude mice. Upper panels show 3 representative tumors from untreated animals, while the lower panels show the tumor inoculation sites in those animals treated with 30 mg/kg×9 doses of ATT-11E. As shown, ATT-11E exerted a potent anti-cancer effect, causing tumor regression and tumor growth inhibition;

FIGS. 12A-B show no detectable adverse effect of several different doses of ATT-11E on body weight in the melanoma A375 tumor-bearing mice. Tumor-bearing mice which were treated with 9 doses of 1, 3, 9 and 20 mg/kg ATT-11E (FIG. 12A) and 6 doses of 40 mg/kg and 9 doses of 20 mg/kg ATT-11E (FIG. 12B) and were weighed twice a week. No discernable effect on animal body weight was shown for any of the doses of ATT-11E.

FIG. 13 shows the results of an experiment testing the maximal tolerated dose (MTD) of ATT-11E in nude mice. Increasing doses of ATT-11E were tested on nude mice for their effect on body weight. MTD was defined as the maximum dose that caused no drug-related lethality and that produced animal body weight loss of <20% of original weight. ATT-11E was administered i.v on 5 consecutive days. Body weight of mice was monitored daily and MTD was determined 24 hr after the last dose. The results show that in this experiment MTD of ATT-11E was not reached, i.e., these doses do not affect survival and does not cause body weight loss, indicating that MTD of ATT-11E is greater than 40 mg/kg. Thus, ATT-11E does not affect animal body weight at doses up to 40 mg/kg;

FIG. 14 shows the efficacy of ATT-11TBE (a compound according to another embodiment of the invention) as a cytotoxic anti-cancer drug, as exemplified in cultured A375-melanoma cells in vitro, in comparison with the anti-cancer drug CPT-11 (Irinotecan). CPT-11 is a pro-drug of SN-38, an anti-cancer agent, active as an inhibitor of the enzyme topoisomerase I. A375 cells were incubated with increasing concentrations of ATT-11TBE or CPT-11, and cell viability was assessed. The cytotoxic effect of these agents was expressed as the percentage of viable cells at 24 hours of incubation. ATT-11TBE exhibited potent cytotoxic effect, with IC₅₀ (inhibitory concentration of 50% of cells) of 0.8 μM, being more potent than CPT-11. Therefore, as shown, ATT-11TBE is a potent anti-cancer agent;

FIG. 15 shows the performance of ATT-11TBE in inhibiting tumor growth in tumor-bearing nude mice. A375 melanoma tumor-bearing mice were treated with ATT-11 TBE (9 doses of 5 or 20 mg/kg), and tumor volumes (mm³) were measured, in comparison with the tumor volumes in control, untreated mice. ATT-11TBE exerted a dramatic effect on the tumors, with tumor regression and growth inhibition, observed with both doses of the drug;

FIG. 16 demonstrates tumor growth delay, exerted by ATT-11TBE in melanoma A375 tumor-bearing mice. Tumor-bearing mice were treated with ATT-11TBE (20 mg/kg), or CPT-11 (75 mg/kg), and tumor growth was followed for 22 days after the last dose. As shown, ATT-11TBE substantially inhibited tumor growth, even 22 days after the last dose, as compared with CPT-11;

FIG. 17 shows lack of adverse effect of ATT-11TBE on body weight in the melanoma A375 tumor-bearing mice. Tumor-bearing mice were treated with ATT-11TBE and were weighed twice a week. As shown, concurrently with the substantial tumor inhibitory effect of ATT-11TBE as described above, the drug was well-tolerated, without causing loss of body weight;

FIG. 18 shows the pharmacokinetic profile of ATT-11TBE, as compared to CPT-11 in beagle dogs. FIG. 18 shows the plasma concentrations of the common active cytotoxic metabolite SN-38 derived from ATT-11TBE or CPT-11 respectively vs. time. As shown, ATT-11 TBE was characterized by favorable profile, with sustained plasma levels and slow clearance of its active metabolite SN-38, as compared to CPT-11;

FIG. 19 shows the plasma half-life (t_{1/2}) of the active cytotoxic metabolite SN-38, derived from ATT-11TBE or CPT-11 respectively, following intravenous drug administration to dogs. SN-38 derived from ATT-11TBE manifested a 3.1-fold longer plasma half-life, as compared to SN-38 derived from CPT-11;

FIG. 20 shows the area under the plasma concentration/time curve (AUC) of the common active cytotoxic metabolite SN-38 derived from ATT-11TBE or CPT-11 respectively, following intravenous drug administration to beagle dogs. AUC of SN-38 derived from ATT-11TBE was 3.6-fold larger than the AUC of SN-38 derived from CPT-11. Therefore, administration of ATT-11TBE resulted in a substantially longer exposure to the common active metabolite SN-38, as compared to CPT-11.

### DETAILED DESCRIPTION OF THE INVENTION

Novel compounds and pharmaceutical compositions containing these compounds are provided. According to some embodiments novel conjugates are provided.

Additional aspects of the invention relate to uses of the compounds and/or conjugates to achieve effective performance of cytotoxic drugs in vivo.

Compounds according to embodiments of the invention may be described by the general formula (I):

Z-L-D (Formula I)

wherein D is preferably a drug, such as a cytotoxic agent; L is either null (absent) or a linker, being either stable or cleavable in physiological conditions; and Z is a modulating moiety.

According to some embodiments of the invention the drug (D in Formula I and/or in the Formulae below) is a cytotoxic anti-cancer agent. Preferably, D is selected from the following groups: alkylating agents (e.g., a nitrosurea drug such as CCNU or BCNU); topoisoemerase I inhibitors (e.g., an Irinotecan, such as camptothecin and its analogues and topotecan); topoisomerase II inhibitors (e.g., doxorubicin); antimetabolites (e.g., a pyrimidine analogue such as 5-fluorouracil); purine analogues (e.g., cladribine); vincristine, taxanes (e.g., paclitaxel), etoposide, platinum agents (e.g., cisplatin, carboplatin); tyrosine kinase inhibitors (e.g., Imatinib); hormone agents (e.g., antiestrogen agents such as Tamoxifen).

According to one embodiment the modulating moiety (Z) is a chemical radical having the following formula (II):

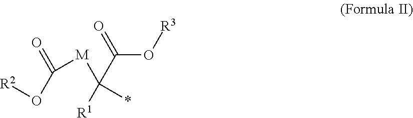

wherein R¹, R², R³ are each independently selected from hydrogen, C₁, C₂, C₃, C₄, C₅, C₆, C₇, C₈ linear or branched alkyl, aryl or heteroaryl, composed of one or two rings, or combinations thereof; R¹, R², R³ may be same or different; M may be absent or may be C₁, C₂, C₃, or C₄ alkylene; and * indicates the linkage point to L or to D, if L is null.

In a preferred embodiment, M is null.

In another preferred embodiment, R¹ is a methyl group.

In another preferred embodiment, R² and R³ are each independently selected from hydrogen, C₁, C₂, C₃, C₄, C₅, or C₆ alkyl groups.

In yet another preferred embodiment, R² and R³ are each an ethyl group. In yet another preferred embodiment, R² and R³ are each a hydrogen atom.

In another preferred embodiment R² is hydrogen and R³ is an ethyl group.

According to one embodiment R² and R³ are each a branched alkyl, such as tert-butyl.

Additionally, compounds such as those described in U.S. Pat. No. 7,270,799 ('799 patent) to Ziv et al., may be used as modulating moieties according to embodiments of the invention. The compounds of the '799 patent, pharmaceutical compositions and methods of their preparation are disclosed in U.S. Pat. No. 7,270,799, which is hereby incorporated by reference.

A compound according to one preferred embodiment is represented by the structure as set forth in Formula (III):

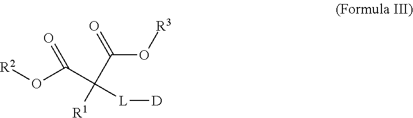

wherein R¹, R² and R³ may each be independently selected from hydrogen, C₁, C₂, C₃, C₄ linear or branched alkyl; R¹, R² and R³ may be the same or different. L is either null or selected from C₁, C₂, C₃, C₄, C₅, C₆, C₇, C₈ linear or branched alkyl, substituted or unsubstituted, alkylene, aryl or heteroaryl, composed of one or two rings, carbamate, amide or ester or combinations thereof; D is preferably a drug, such as a cytotoxic agent. In one embodiment of the invention D in Formula III is a camptothecin analogue, or nitrosurea.

A compound according to a further embodiment of the present invention is represented by the structure as set forth in Formula IV.

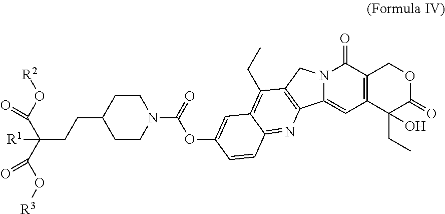

wherein R¹, R² and R³ are each independently selected from hydrogen, or C₁, C₂, C₃, C₄ linear or branched alkyl; R¹, R² and R³ may be the same or different.

In one preferred embodiment R² is hydrogen and R³ is an ethyl group.

According to yet another specific embodiment, Formula IV includes a camptothecin analogue and a monoester modulating moiety (malonate monoester) attached by a linker (see Formula V below).

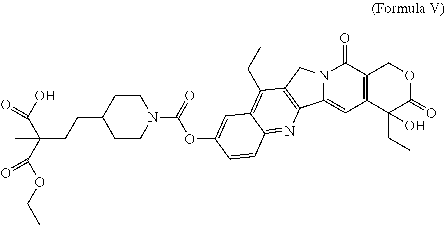

In one specific embodiment, Formula IV includes a compound named ATT-11E (see Formula VI below). ATT-11E includes a camptothecin analogue (SN-38, which may be released in vivo due to cleavage of ATT-11E by carboxyl esterases) and an esteric modulating moiety (malonate ester) attached by a linker.

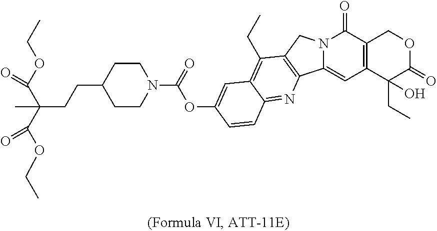

In another specific embodiment Formula IV includes a compound named ATT-11 TBE (see Formula VI′ below). ATT-11TBE includes the camptothecin analogue SN-38 and a tert-butyl malonate ester moiety attached by a linker.

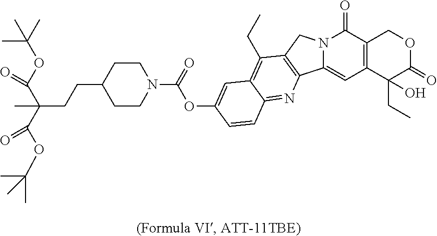

According to yet another embodiment of the invention, there is provided a compound represented by the structure as set forth in Formula VII:

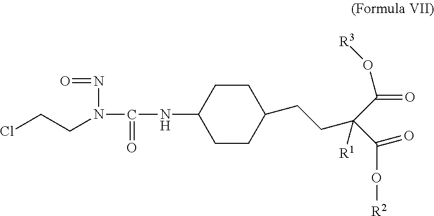

wherein R¹ and R², R³ are each independently selected from hydrogen, C₁, C₂, C₃, C₄, C₅, linear or branched alkyl; R¹, R², and R³ may be the same or different.

In one specific embodiment Formula VII includes a compound named ATT-1 (see formula below). ATT-1 includes CCNU, a nitrosurea anticancer chemotherapy drug, attached to a modulating moiety (pentyl malonic acid).

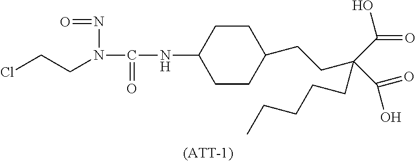

In a second embodiment, Formula VII includes a compound named ATT-5E, (see formula below). ATT-5E includes CCNU attached to a modulating moiety (pentyl malonate ester) (an esteric modulating moiety may confer a different bio-distribution pattern than a

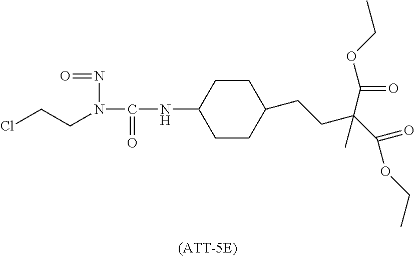

In these specific embodiments the nitrosurea anticancer chemotherapy drug used is CCNU however, it should be realized that other nitrosurea drugs may be used in similar embodiments.

The compounds of the invention (for example, compounds of Formulae III-VII) may also be viewed as conjugates (as defined hereinabove), which comprise a cytotoxic agent and a modulating moiety, which may modulate the performance of the cytotoxic drug upon administration.

According to one embodiment, the modulating moiety according to embodiments of the invention, targets the compounds to cells undergoing cell death, for example, within a tumor. Tumor tissue is often characterized by an increased number of cells undergoing cell death (apoptosis), as compared to normal tissue, as the result of the relative fragility of cancer cells, or hypoxic or ischemic conditions characterizing the tumor. Compounds according to embodiments of the invention may selectively target dying (apoptotic) cells at multiple foci within a tumor. According to one embodiment, due to the modulating moiety, the compounds may penetrate apoptotic cells and may be retained in the cells essentially creating a depot of cytotoxic agents. This depot may enable high concentration and long residence time of the cytotoxic agents within the tumor. The cytotoxic agents are then released into the extra-cellular space and tissue surrounding the apoptotic cells in the tumor, thus being able to contact and destroy viable tumor cells.

The compounds of the present invention may act as pro-drugs, wherein when the compounds are intact (such as a compound having the general formula as described by Formula I or III) the drug or cytotoxic agent is inactive, whereas upon cleavage of the compound, an active cytotoxic element is released.

A compound according to embodiments of the invention may be advantageous over an un-conjugated drug in one or more aspects of performance, such as, for example, efficacy, toxicity, or pharmacokinetics. For example, the pattern of distribution of an anticancer agent upon its systemic administration may be modulated by the compounds according to embodiments of the invention. Examples for such modulations are, without limitation, enhancement of drug levels within a tumor relative to its levels within non-target tissues, or modulation of absorption, metabolism, excretion pattern of the drug and/or other pharmacokinetic features.

According to embodiments of the invention a modulating moiety (e.g., Z in Formula I) is an apoptosis specific marker, namely, a compound capable of targeting an apoptotic cell. According to embodiments of the invention an apoptosis specific marker may be conjugated to an appropriate cytotoxic drug, with or without a linker, to improve performance of the drug, for the treatment of cancer or for modulation of the immune system. According to other embodiments of the invention, the modulating moiety may be specific to other cell types and may have other modulating properties.

Embodiments of the invention provide a pharmaceutical composition comprising a compound or conjugate as described above (for example as described by Formulae III-VII) and/or as described in the Examples below, and pharmaceutically acceptable salts, hydrates and solvates thereof and solvates and hydrates of the salts. Some examples of salts include nontoxic alkaline metal salts, alkaline earth metal salts and ammonium salts such as sodium, potassium, lithium, calcium, magnesium, barium and ammonium salts. In addition, nontoxic acid addition salts are also included in the above-mentioned salts, for example, hydrochlorides, hydrogen chlorides, hydrogen bromides, sulfates, bisulfates, acetates, oxalates, valerates, oleates, laurates, borates, benzoates, lactates, malates, p-toluene sulfonates (tosylates), citrates, maleates, fumarates, succinates, tartrates, sulfonates, glycolates, maleates, ascorbates and benzene sulfonates.

Pharmaceutical compositions according to embodiments of the invention may include a pharmaceutically accepted carrier such as a diluent, adjuvant, excipient, or vehicle with which the therapeutic agent is administered. Some examples of pharmaceutically acceptable carriers include water, salt solutions, alcohol, silicone, waxes, petroleum jelly, vegetable oil, peanut oil, soybean oil, mineral oil, sesame oil, polyethylene glycols, propylene glycol, liposomes, sugars, gelatin; lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The pharmaceutical compositions may be manufactured, for example, by means of conventional mixing, dissolving, granulating, levitating, emulsifying, encapsulating, entrapping, lyophilizing processes or other suitable processes.

The pharmaceutical compositions can take the form of solutions, suspensions, emulsions, tablets, pills, capsules, powders, sustained-release formulations and the like, depending on the intended route of administration. Examples of routes of administration include parenteral, e.g., intravenous, intramuscular, subcutaneous, intra-arterial, intraportal, intrathecal, intradermal, transdermal (topical), transmucosal, intra-articular, intraperitoneal, and intrapleural, as well as intrathecal, intracerebral, inhalation and pulmonary administration. In another aspect, the delivery system and pharmaceutical composition are administered to the subject locally, for example, by injection to a local blood vessel which supplies blood to a particular tumor, organ, tissue, or cell afflicted by disorders or diseases.

The pharmaceuticals according to embodiments of the invention can be administered orally, for example in the form of pills, tablets, lacquered tablets, sugar-coated tablets, granules, hard and soft gelatin capsules, aqueous, alcoholic or oily solutions, syrups, emulsions or suspensions. For the production of pills, tablets, sugar-coated tablets and hard gelatin capsules it is possible to use, for example, lactose, starch, for example maize starch, or starch derivatives, talc, stearic acid or its salts, etc. Carriers for soft gelatin capsules and suppositories are, for example, fats, waxes, semisolid and liquid polyols, natural or hardened oils, etc. In one embodiment, the medicament, together with one or more auxiliary excipient materials may be compressed into a tablet form such as a single layer or multilayer tablet. Tablets according to embodiments of the invention can optionally be coated with a controlled release polymer so as to provide additional controlled release properties.

For example, for parenteral administrations, the composition may comprise one or more of the following components: a sterile diluent such as water for injection, saline solution; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic.

For example, for injection, the conjugates of the invention may be formulated in aqueous solutions, preferably in physiologically compatible buffers such as physiological saline buffer. The solution may contain formulatory agents such as suspending, stabilizing and/or dispersing agents. In a preferred embodiment, the delivery systems are formulated in sterile aqueous solutions.

For example, for intravenous administration, suitable carriers include physiological saline, bacteriostatic water, or phosphate buffered saline (PBS). In all cases, the composition must be sterile and, when injected, should be fluid to the extent that easy injectability with a syringe. Compositions may include preservatives such as, parabens, chlorobutanol, phenol, ascorbic acid, thimerosal, and the like.

For example, for administration by inhalation, the delivery systems may be formulated as an aerosol spray from pressurized packs or a nebulizer, with the use of a suitable propellant, e.g., dichlorodifluoromethane, trichlorofluoromethane, dichlorotetrafluoroethane, carbon dioxide or other suitable gas.

Various doses can be used, varying according to the disease, the clinical status of the patient or concomitant medications. Dosage can vary, according to the clinical judgment of the physician.

Embodiments of the invention provide methods for treatment of a medical disorder. According to an embodiment of the invention, one method comprises administering to a patient or subject a pharmaceutically effective dose of a conjugate according to embodiments of the invention. The conjugate may be administered with additives such as described above. Administration may take any suitable route, such as described above.

The medical disorder may be, inter alia, a neoplastic disease, i.e., cancer and/or an immune-mediated disease. Said cancer or immune-mediated disease may involve or originate from any organ in the body.

In a preferred embodiment, the type of cancer to be treated by the compounds of the invention and/or their related pharmaceutical compositions includes primary or secondary tumors of the lung, colon, breast, melanoma, lymphoma, prostate, thyroid, testes, ovary, skin, brain or bone.

According to some embodiments of the invention, the compounds or their related pharmaceutical compositions may be used as a monotherapy, i.e., as a single therapeutic agent, or in combination with other therapeutic agents, i.e., as part of a combination therapy. Treatment can be acute or chronic.

Some examples will now be described to further illustrate the invention and to demonstrate how embodiments of the invention may be carried-out in practice. In the Examples compounds according to three embodiments of the invention are demonstrated. The demonstrated compounds all conform to Formula I (i.e., include D, L and Z groups as defined in Formula I). More specifically, the demonstrated compounds confer to Formula III, while demonstrating various D, L and Z groups. These Examples demonstrate a variety of similar compounds having in common structural features according to the embodiments of the invention, which, as shown below, enable potent cytotoxic activity, both in vitro and in vivo. Although the Examples relate to specific compounds and to specific conditions (such as specific administration routes) these are intended only to exemplify the invention and not to limit the scope of the invention.

### EXAMPLES

In the Examples, compounds will be shown, as well as exemplary methods for preparation of these compounds and also examples of use of these compounds in vitro and in vivo. The Examples demonstrate compounds and conjugates of the present invention as tumor targeting agents and potent tumor inhibiting agents, having potentially enhanced safety profile compared to known cytotoxic agents.

### Exemplary Compounds

Four exemplary compounds are demonstrated, all of which conform to the general formula of Formula I and are an embodiment of this formula.

Z-L-D (Formula I)
wherein D is a drug, such as a cytotoxic agent; L is either null or a linker, being either stable or cleavable in physiological conditions; and Z is a modulating moiety. According to an embodiment of the invention, L is a linker, selected from C₁, C₂, C₃, C₄, C₅, C₆, C₇, C₈ linear or branched, substituted or unsubstituted, alkylene, aryl or heteroaryl, composed of one or two rings, carbamate, amide or ester or combinations thereof.

In a first compound (termed ATT-1) D is CCNU and Z is pentyl malonic acid. In this embodiment L is null.

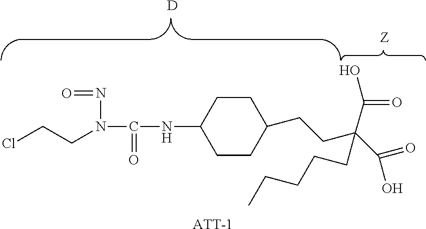

In a second compound (termed ATT-5E) D is CCNU and Z is a malonate ester. In this embodiment component L is null.

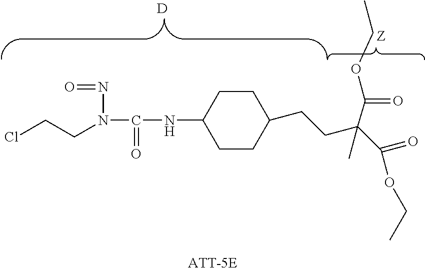

In a third compound (termed ATT-11E) D is a camptothecin analogue, SN-38 and Z is a malonate ester attached by a linker (L).

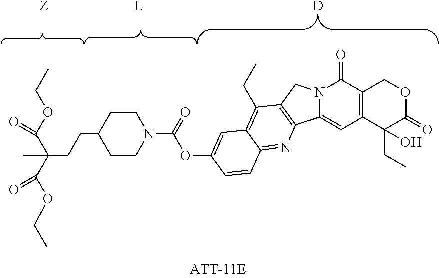

In a fourth compound (termed ATT-11TBE) D is a camptothecin analogue, SN-38 and Z is a tert-butyl malonate ester attached by a linker (L).

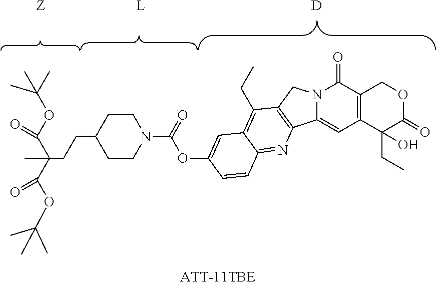

### Exemplary Methods for Preparation

Four exemplary methods are demonstrated. However, modifications of these methods (e.g., similar processes including additional or other trivial steps such as alkylation, hydration, condensation and other well known steps), which may result in similar compounds, are also included in embodiments of the invention.

### Example 1

A synthesis scheme of ATT-1, according to one embodiment, is illustrated below:

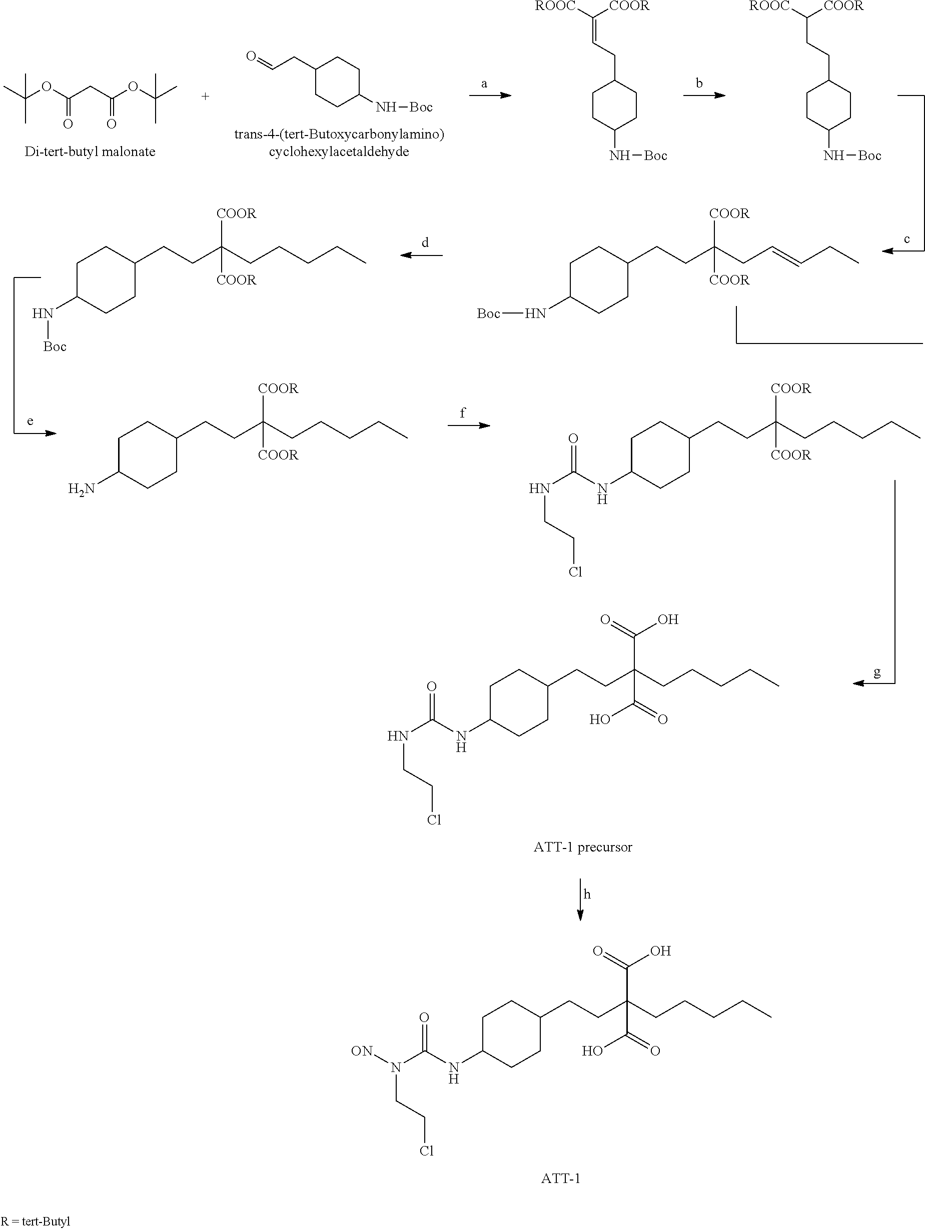

In step (a) a Knoevenagel condensation was performed; pyridine:piperidine (20:1) at 80-90° C. for 8 hours. Step (b) was performed in the presence of H2, 10% Pd/C, MeOH at room temperature for 6 hours. Step (c) was performed in the presence of 1-bromo-2-pentene, DMF/HMDS (0.5M toluene solution) at room temperature for 20 hours. Step (d) was performed in the presence of H2, 10% Pd/C, MeOH at room temperature for 6 hours. Step (e) was performed in the presence of 2N HCl/IPA at room temperature for approximately 15 hours. Step (1) was performed in the presence of 2-chloroethylisocyanate, Et₃N/DCM at room temperature for an hour. Step (g) was performed in the presence of TFA/DCM at room temperature for approximately 2-3 hours. Step (h) was performed in the presence of NaNO₂/HCOOH to obtain ATT-1.

### Example 2

A synthesis scheme of ATT-5E, according to one embodiment, is illustrated below:

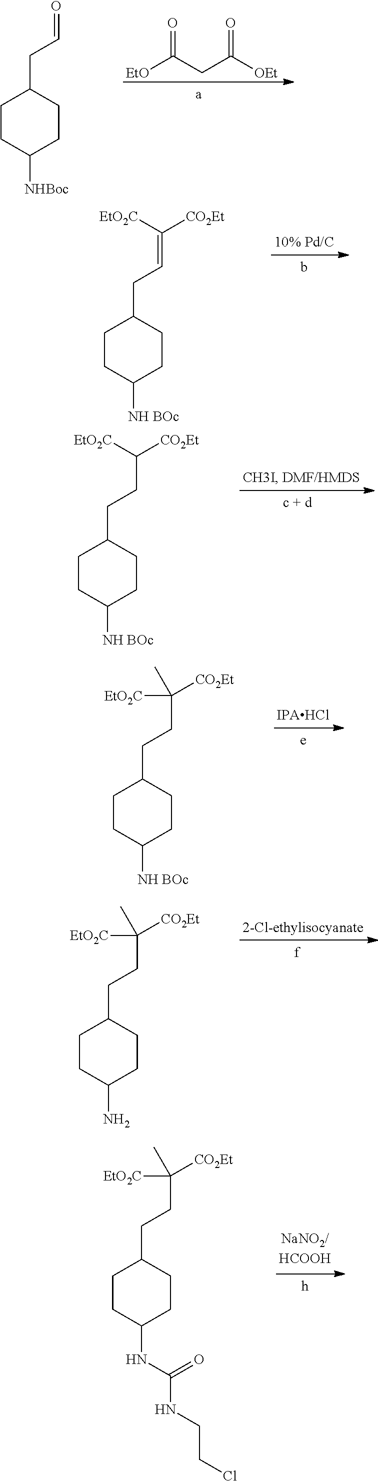

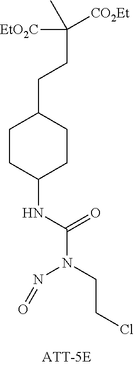

In step (a) a Knoevenagel condensation was performed; pyridine:piperidine (20:1) at 80-90° C. for 8 hours. Step (b) was performed in the presence of H2, 10% Pd/C, MeOH at room temperature for 6 hours. Step (c) was performed in the presence of 1-bromo-2-pentene, DMF/HMDS (0.5M toluene solution) at room temperature for 20 hours. Step (d) was performed in the presence of H2, 10% Pd/C, MeOH at room temperature for 6 hours. Step (e) was performed in the presence of 2N HCl/IPA at room temperature for approximately 15 hours. Step (f) was performed in the presence of 2-chloroethylisocyanate, Et₃N/DCM at room temperature for an hour. Step (h) was performed in the presence of NaNO₂/HCOOH to obtain ATT-5E.

### Example 3

A synthesis scheme of ATT-11E, according to one embodiment of the invention is illustrated below:

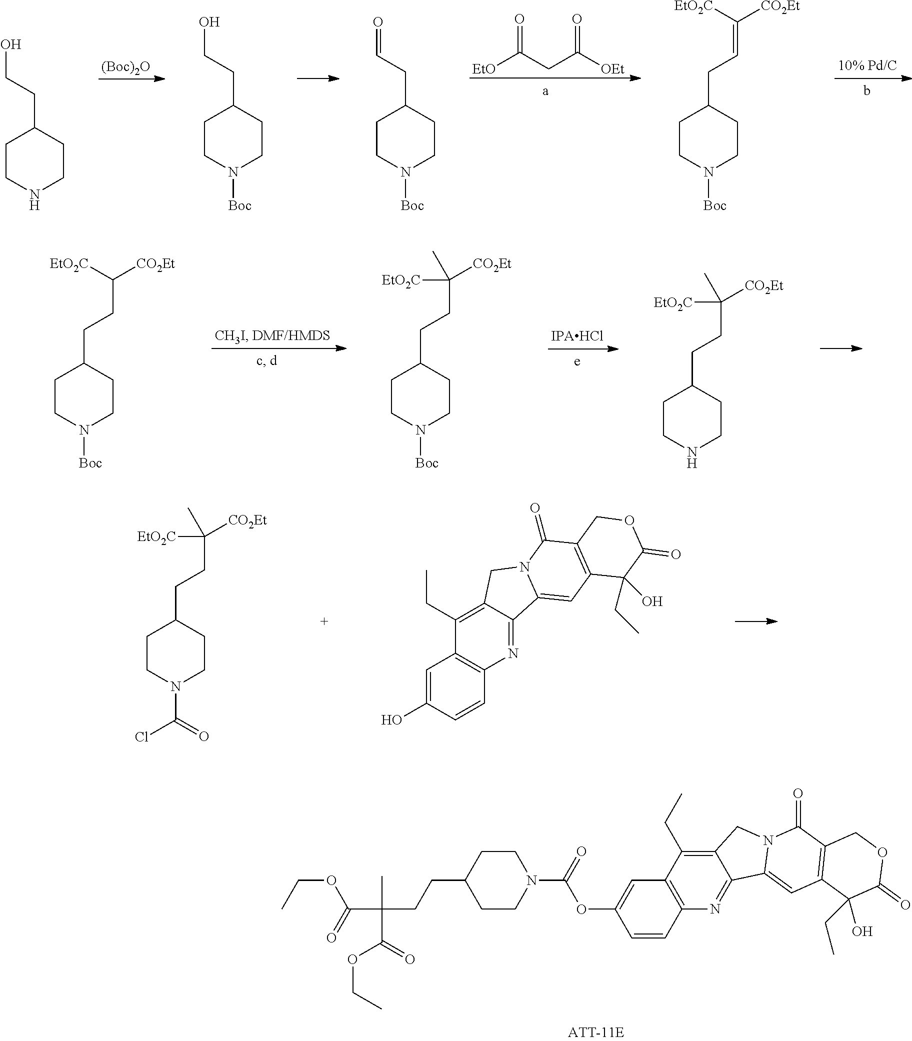

In Step (a) a Knoevenagel condensation was performed; pyridine:piperidine (20:1) at 80-90° C. for 8 hours. Step (b) was performed in the presence of H2, 10% Pd/C, MeOH at room temperature for 6 hours. Step (c) was performed in the presence of 1-bromo-2-pentene, DMF/HMDS (0.5M toluene solution) at room temperature for 20 hours. Step (d) was performed in the presence of H2, 10% Pd/C, MeOH at room temperature for 6 hours. Step (e) was performed in the presence of 2N HCl/IPA at room temperature for approximately 15 hours. SN-38 was linked to the resulting product to obtain ATT-11E.

### Example 4

A synthesis scheme of ATT-11TBE, according to one embodiment of the invention is illustrated below:

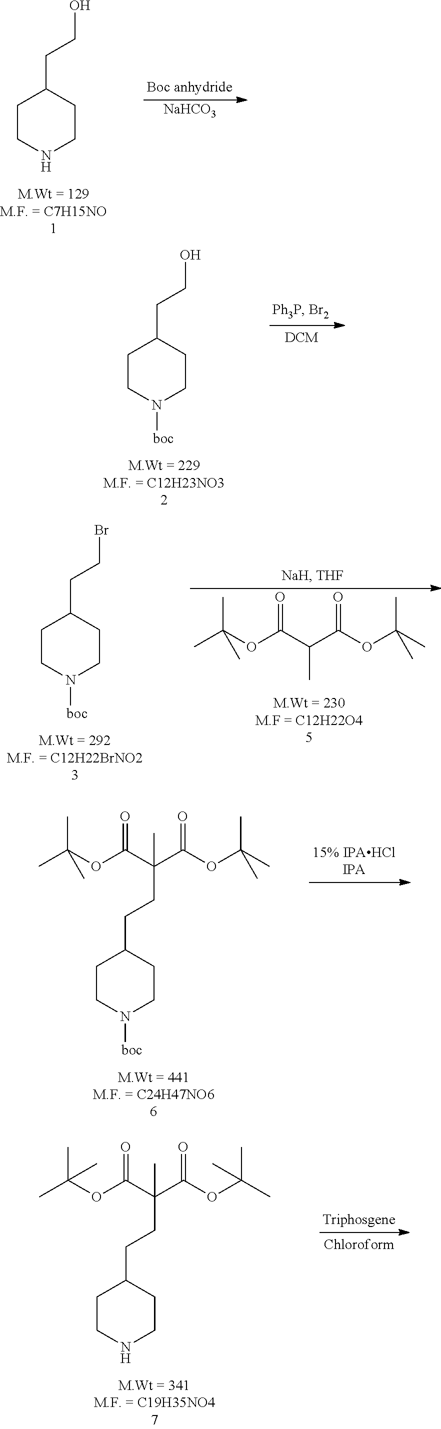

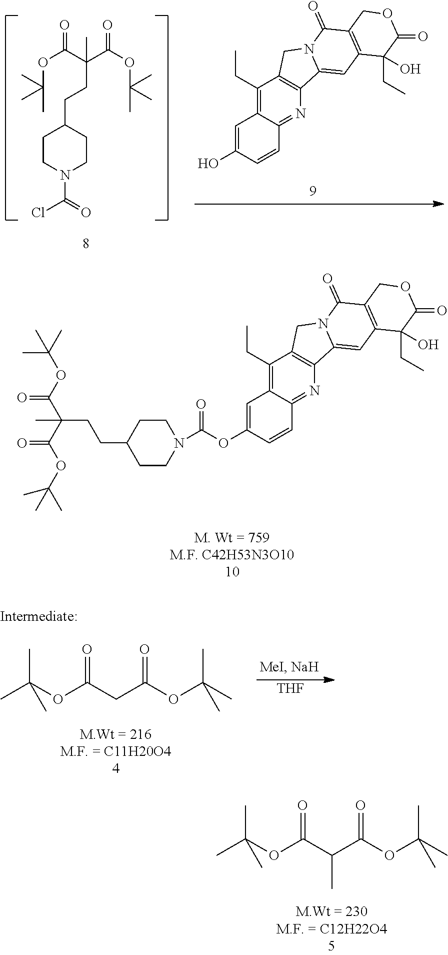

In a first step, Boc protection is preformed on a piperidine derivative 1, followed by bromination to obtain compound 3, and condensation with methyl di tert butylmalonate, which is produced by methylation of di tert butylmalonate 4. Said condensation results in compound 6, which is then Boc-de-protected and condensed with SN-38 (compound 9), to result in compound 10, i.e., the desired product ATT-11TBE. The Examples will further describe experiments which exhibit the performance of several compounds according to embodiments of the invention, as potent anticancer agents, both in vitro, and in vivo, in xenograft animal models of cancer. All experiments were performed in quadruplicate and repeated several independent times. Results are shown as means±SEM.

### Exemplary Uses In Vitro and In Vivo

### Example 5

### This Example Demonstrates the Efficacy of ATT-1 as an Anti-Cancer Drug In Vitro

The efficacy of ATT-1 as an anti-cancer drug is exemplified in cultured A375-melanoma cells in vitro. The cells were grown in RPMI 1640 (Gibco, UK) supplemented with 10% FBS (Gibco, UK) 2 mM of L-glutamin (Gibco, UK), 20 mM HEPES buffer (Beit-Haemek, Israel), 100 units/ml of penicillin and 100 μg/ml of streptomycin (Gibco, UK). Cells were cultured in humidified atmosphere containing 5% CO₂ at 37° C. The medium was routinely changed twice a week and cells were subcultured with 0.25% trypsin/EDTA (Beit-Haemek, Israel) when they reached 85% confluence.

ATT-1 was produced by Aptuit-Laurus (India). ATT-1 was applied to melanoma (A375) cells. The in vitro cytotoxicity was determined using a cell proliferation tetrazolium dye reduction assay. Adherent cells (5×10³ per well) were seeded as monolayer in 96 well plates and incubated overnight at 37° C. The cells were then treated with serial dilutions of ATT-1 dissolved in growing medium (stock solution of ATT-1 was prepared in DMSO) and further incubated for 48 hr at 37° C. followed by cell viability determination. Cell viability was determined using XTT reagent (Beit-Haemek, Israel) according to the manufacturer's instructions. At the end of the incubation period, the tetrazolium dye was added and formation of a colored product, formazan, was measured at 450 nm using microplate reader (ELx800, Bio-Tek instruments Inc.).

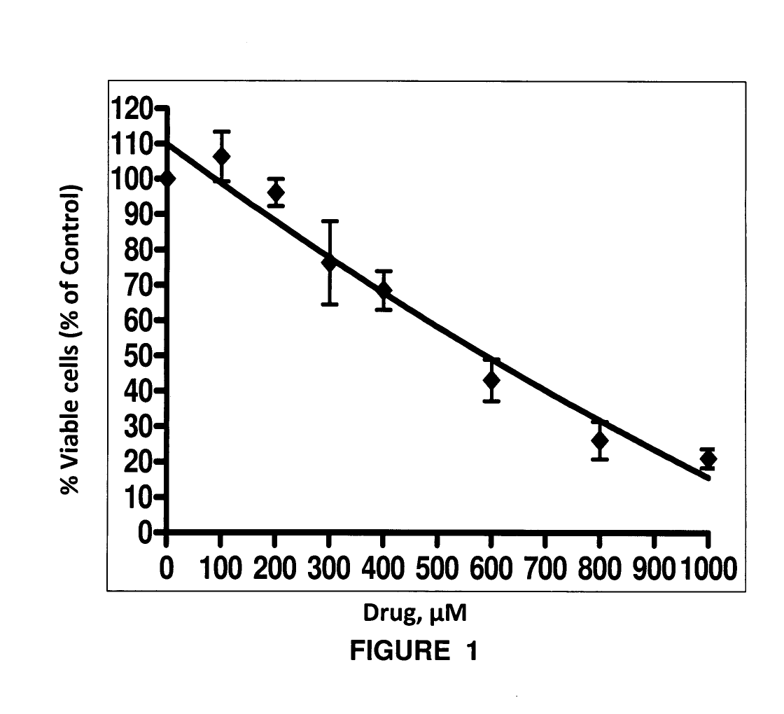

The results, displayed in FIG. 1, show the cell killing effect of ATT-1 on melanoma cells as a percentage of viable cells. A graph showing the percent of viable treated cells presented as a percentage of untreated cells is displayed in FIG. 1. The IC₅₀ value of ATT-1 was calculated as the drug concentration that inhibits 50% of the cell growth as compared with the control. IC₅₀ value was estimated fitting the data with a non-linear regression analysis using GraphPad prisma ver.5.0 software. ATT-1 exhibited an IC₅₀ of 545 μM. Thus, a clear killing effect and cytotoxic performance of ATT-1 is demonstrated in vitro.

### Example 6

### This Example Demonstrates the Efficacy of ATT-1 in Inhibiting Tumor Growth In Vivo

To evaluate the effect of ATT-1 on tumor growth in vivo, tumor volume was measured regularly during ATT-1 treatment. Subcutaneous melanoma tumors were established by injection of 0.75×10⁶ A375 cells into the right flank region of athymic nude mice (female, 8-9 weeks). Treatment, with ATT-1 administered in multiple doses, was initiated when tumors reached an average volume of 75 to 100 mm³. Tumors were measured twice each week with a caliper and the tumor volume (TV) was calculated according to the formula: TV=0.52 L×W², where L and W are the major and minor dimensions, respectively. Control tumors reached a predetermined volume limit, after which the animals were sacrificed.

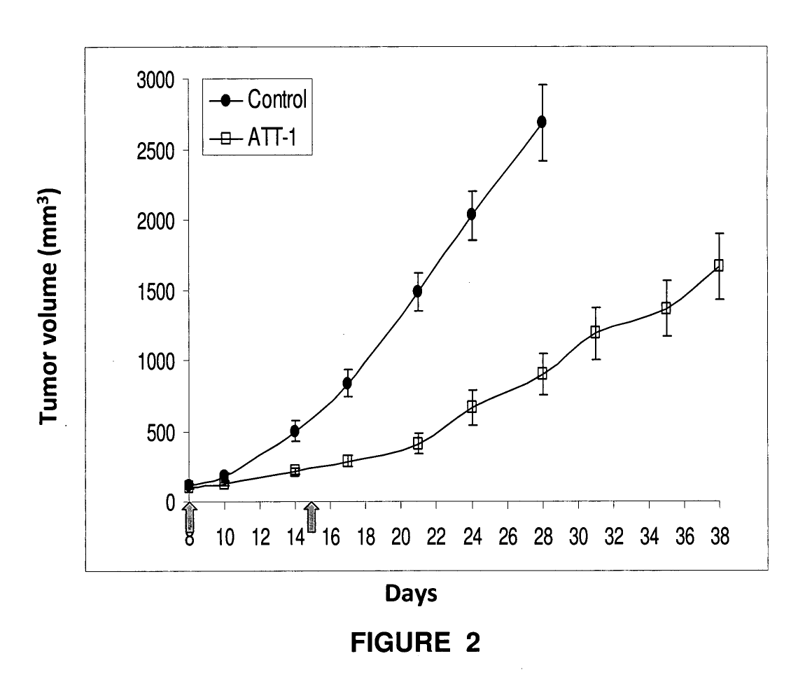

In a first experiment (FIG. 2), ATT-1 was administered to the mice (8-9 mice/group) in two doses. A first dose of ATT-1 at 50 mg/kg was administered on day 8 (post tumor cell inoculation). A second dose of 40 mg/kg was administered on day 15. The efficacy of the drug treatment assessed as TVI (%)-percentage of tumor volume inhibition in treated versus control tumors. TVI (%) was calculated according to the equation: TVI(%)=(1-TIC)×100, where T and C were the tumor volume of the treated and control groups, respectively.

The results of the first experiment are displayed in FIG. 2 as tumor volume (mm³) per day of treatment. Arrows on the graph in FIG. 2 mark days of administration. Tumor growth inhibition by ATT-1 as compared to the control was observed. At day 28, control tumors reached a predetermined volume limit and the ratio of tumor volume (mm³) of treated compared to control (T/C) was 0.33 (p<0.001). Tumor growth inhibition of 77% compared to the control was displayed demonstrating a clear effect of ATT-1 on tumor growth.

In a second experiment the inhibitory effect of ATT-1 on tumor growth compared to CCNU at equal total dose, was demonstrated.

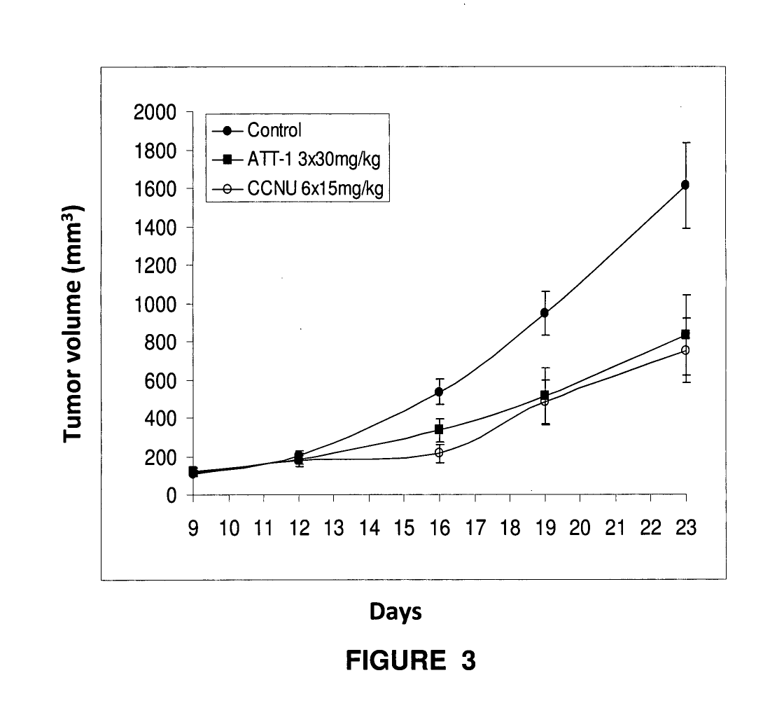

ATT-1 (3 doses of 30 mg/kg) and CCNU (6 doses of 15 mg/kg) were administered to mice as described above. Individual doses of CCNU were reduced to 15 mg/kg because of the high toxicity of CCNU to healthy tissue. The results, which are displayed in FIG. 3 as tumor volume (mm³) per day of treatment, show that at equal total doses, both ATT-1 and CCNU inhibit tumor growth (CCNU-53% inhibition, ATT-1-48% inhibition). Tumor volume (mm³) of CCNU treated compared to control (T/C) at day 23 is 0.47. Tumor volume (mm³) of ATT-1 treated compared to control (T/C) at day 23 is 0.52 (P_{Control}<0.05). Thus, a similar inhibitory effect on tumor growth is exhibited for ATT-1 and CCNU, demonstrating that ATT-1 is as potent as CCNU in tumor growth inhibition.

A third experiment demonstrates the inhibitory effect of ATT-1 on tumor growth compared to CCNU at equi-molar doses.

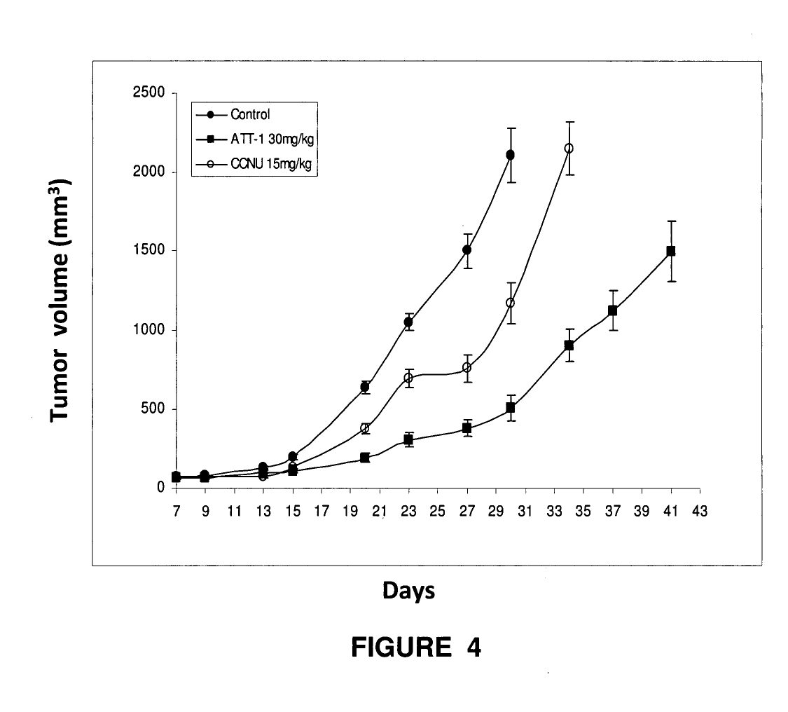

In order to compare equivalent molar doses of ATT-1 and CCNU the effect of ATT-1 (3×30 mg/kg) and CCNU (3×15 mg/kg) on melanoma (A375) tumor growth was measured as described above. The results, which are displayed in FIG. 4 as tumor volume (mm³) per day of treatment, show that on the basis of equi-molar doses a marked tumor growth delay was demonstrated by ATT-1 in comparison with CCNU (76% growth inhibition of ATT-1 vs. 48% growth inhibition by CCNU). T/C at day 30 for ATT-1 was 0.24 compared to 0.56 for CCNU. This experiment demonstrates a greater effect on tumor growth inhibition of ATT-1 as compared to equimolar CCNU.

### Example 7

### This Example Evaluates Potential Adverse Effects on Body Weight of ATT-1 in Tumor-Bearing Nude Mice

For the evaluation of potential adverse effects of ATT-1, nude mice, bearing the A375 melanoma tumors and treated with ATT-1 as described above, were monitored for adverse effects immediately after drug administration, and twice a week, through one day after last dose. The evaluation protocol consisted of gross observation, registration of abnormal signs, and determination of body weight. Potential body weight loss (BWL) was calculated as % BWL=100-(BW_{dayx}/BW_{day1}×100), where day 1 is the first day of treatment and day x is the day of assessment thereafter.

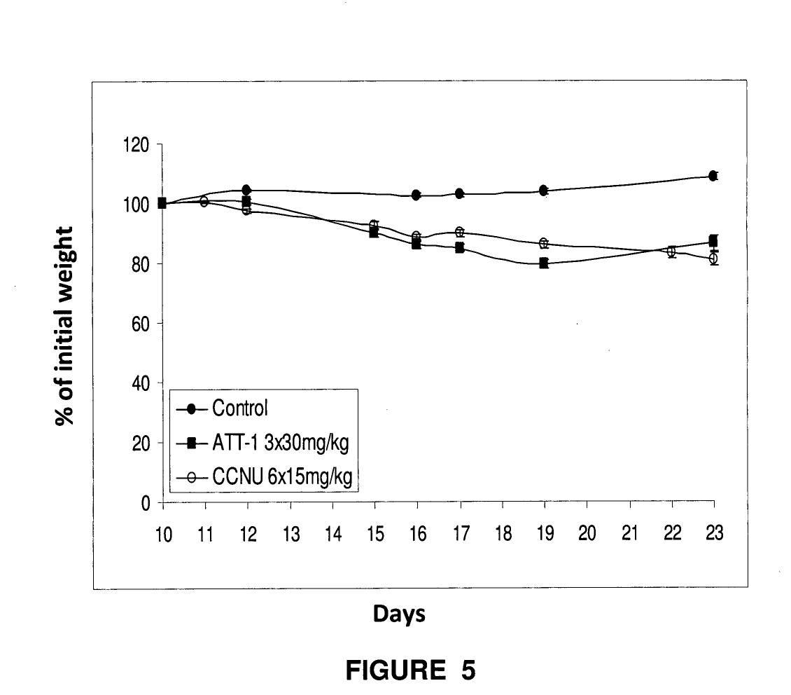

ATT-1 (3 doses of 30 mg/kg) and CCNU (6 doses of 15 mg/kg) were administered to A375 tumor bearing mice. The results are displayed in FIG. 5 in terms of % of initial weight per day starting at day 10 after initiation of treatment. At day 23 ATT-1 treated animals showed a weight loss of 14% compared to a weight loss of 19% demonstrated for CCNU treated animals. Thus, it can be seen that the effect of ATT-1 on body weight is similar, or in the same magnitude of the effect of CCNU.

### Example 8

### This Example Evaluates Potential Adverse Effects on White Blood Cell (WBC) Count of ATT-1 in Tumor-Bearing Nude Mice

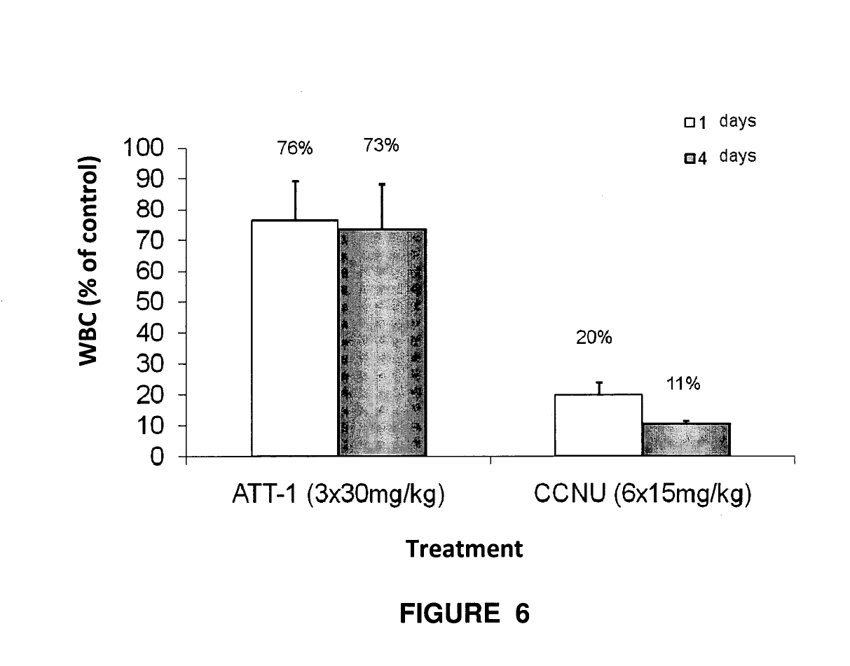

Repeated doses of ATT-1 (3 doses of 30 mg/kg) and CCNU (6 doses of 15 mg/kg) were administered to A375 tumor bearing mice and WBC was tested (at AML, Herzelia, Israel) 1 and 4 days post last dose. The results are displayed in FIG. 6, showing the percent of WBC compared to the control for ATT-1 treated animals and CCNU treated animals on day 1 and day 4. A markedly lower affect on WBC was demonstrated for ATT-1 treated animals than for CCNU treated animals (27% reduction of WBC for ATT-1 vs. 89% reduction of WBC for CCNU). These results indicate that ATT-1 treatment preserves white blood cell count and does not cause a reduction of white blood cells.

### Example 9

### This Example Demonstrates the Efficacy of ATT-5E as an Anti-Cancer Drug In Vitro

The efficacy of ATT-5E as an anti-cancer drug is exemplified in cultured

A375-melanoma cells in vitro, in comparison with the commercially available anti-cancer drug CCNU. ATT-5E was produced by Aptuit-Laurus (India).

A375 cells were incubated with increasing concentrations of either ATT-5E or CCNU, cell viability was evaluated, and IC₅₀ (inhibitory effect of 50% cells) was calculated.

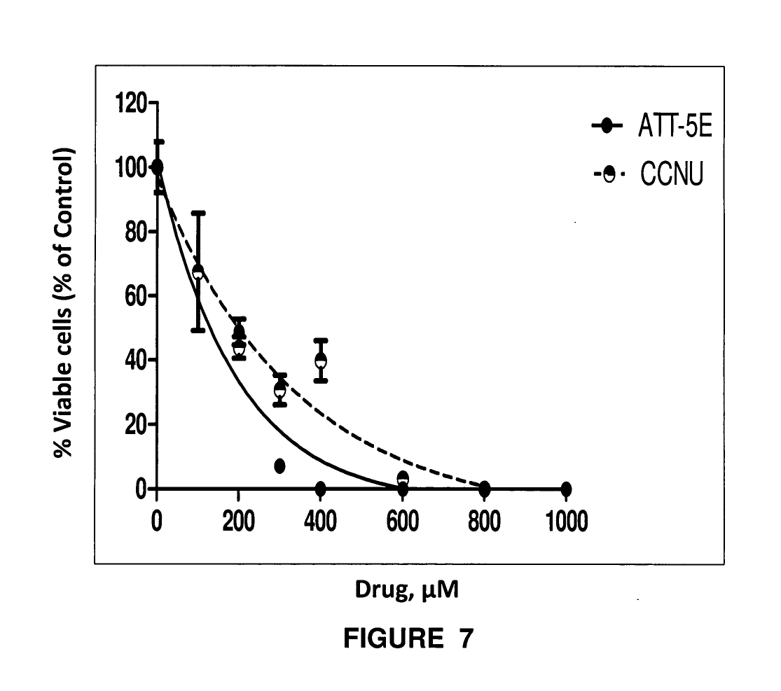

The in vitro cytotoxicity was determined using a cell proliferation tetrazolium dye reduction assay. Adherent cells (5×10³ per well) were seeded as monolayer in 96 well plates and incubated overnight at 37° C. The cells were then treated with serial dilutions of ATT-5E in growing medium (stock solution of ATT-5E was prepared in DMSO) and further incubated for 48 hr at 37° C. followed by cell viability determination. Cell viability was determined using XTT reagent (Beit-haemek, Israel) according to the manufacturer's instructions. At the end of the incubation period, the tetrazolium dye was added and formation of a colored product, formazan, was measured at 450 nm using microplate reader (ELx800, Bio-Tek instruments Inc.). The results, displayed in FIG. 7, show the cell killing effect of ATT-5E and CCNU on melanoma cells as a percentage of viable cells as compared to control cells for increasing concentrations of ATT-5E and CCNU. The IC₅₀ value of ATT-5E (calculated as described above) was 130 μM whereas CCNU exhibited an IC₅₀ of 200 μM. These results indicate a higher cell killing effect of ATT-5E compared to CCNU.

### Example 10

### This Example Demonstrates the Efficacy of ATT-11E as an Anti-Cancer Drug In Vitro

The efficacy of ATT-11E as an anti-cancer drug is exemplified in cultured A375-melanoma cells in vitro, in comparison with the commercially available anti-cancer drug Irinotecan (CPT-11). Irinotecan is a pro-drug of SN-38, an anti-cancer agent, active as an inhibitor of the enzyme topoisomerase I. ATT-11E (produced by Aptuit-Laurus (India)) and Irinotecan (CPT-11 obtained from Pfizer) were applied to melanoma (A375) cells. ATT-11E was dissolved in phosphate buffer (pH 7.4) containing 10% DMSO and 30% cremophor and administered by i.v. injection.

The in vitro cytotoxicity was determined using a cell proliferation tetrazolium dye reduction assay. Adherent cells (5×10³ per well) were seeded as monolayer in 96 well plates and incubated overnight at 37° C. The cells were then treated with serial dilutions of ATT-11E in growing medium (stock solution of ATT-11E was prepared in DMSO) and further incubated for 24 hr at 37° C. followed by cell viability determination. Cell viability was determined using XTT reagent (Beit-Haemek, Israel) according to the manufacturer's instructions. At the end of the incubation period, the tetrazolium dye was added and formation of a colored product, formazan, was measured at 450 nm using microplate reader (ELx800, Bio-Tek instruments Inc.)

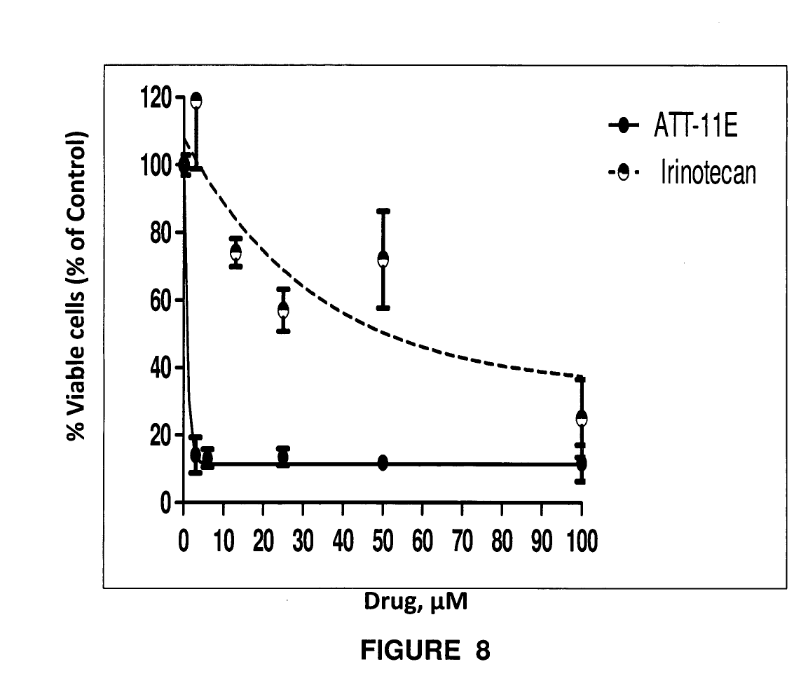

The results, displayed in FIG. 8, show the cell killing effects of ATT-11E and Irinotecan on melanoma cells as a percentage of viable cells as compared to control cells for increasing amounts of ATT-11E and Irinotecan. A graph showing the killing effect of ATT-11E compared to the effect of Irinotecan in terms of percent of viable cells, is displayed in FIG. 8. ATT-11E exhibited an IC₅₀ of 0.6 μM whereas Irinotecan exhibited an IC₅₀ of 44 μM. Thus, a clear cell killing effect of ATT-11E and a dramatically higher cytotoxicity of ATT-11E compared to Irinotecan was shown in vitro.

### Example 11

### This Example Demonstrates the Efficacy of ATT-11E in Inducing Tumor Regression and in Inhibiting Tumor Growth in a Xenograft Model of Melanoma in Nude Mice In Vivo

Subcutaneous A375 melanoma tumors were established in mice by injection of A375 melanoma cells (0.75×10⁶ per animal per site) into the right flank region of athymic nude mice (female, 8-9 weeks, 10 animals per group). Tumor dimensions were measured with a caliper twice each week and tumor volume (mm³) was calculated using the Formula: TV=0.52 L×W², where L and W are the major and minor dimensions, respectively. ATT-11E was administered to the mice (10 mice/group) by two different regimen routes. Regimen I includes 9 doses of 20 mg/kg ATT-11E administered 3 times a week on days 10, 14, 16, 18, 21, 23, 25, 28 and 30 post tumor cells inoculation. Regimen II includes 6 doses of 40 mg/kg ATT-11E administered twice a week on days 10, 14, 17, 21, 24 and 28 post tumor cell inoculation. Control mice were treated with the vehicle solution. The efficacy of the drug treatment assessed as inhibition of tumor growth (TVI (%)) in treated versus control tumors was calculated according to the equation: TVI(%)=(1−T/C)×100, where T and C were the tumor volume of the treated and control groups, respectively.

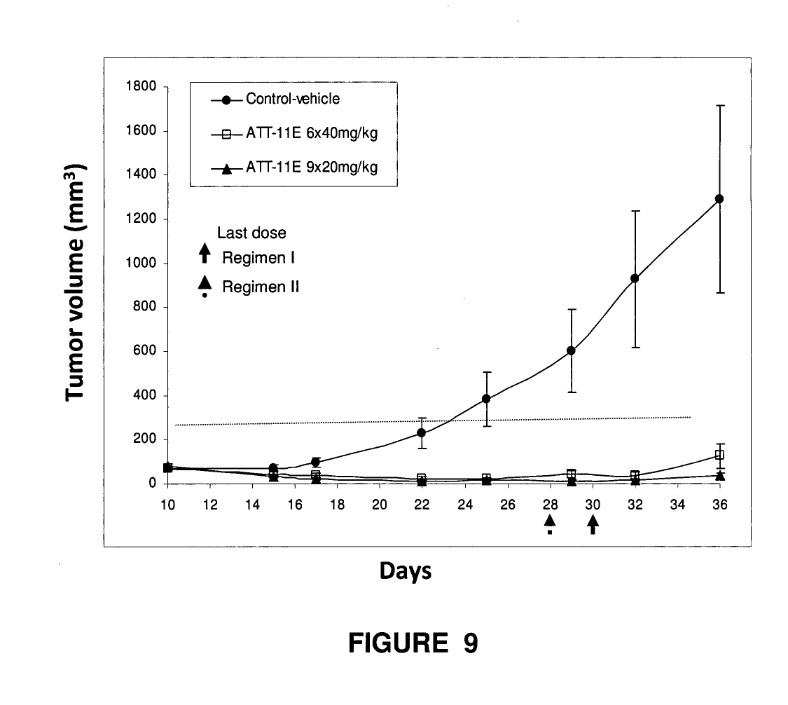

The results displayed in FIG. 9 depict tumor volume (mm³) per day of treatment. An arrow marks the last day of treatment for each regimen. In both regimens, a dramatic inhibition of tumor volume was exhibited (97% tumor volume growth inhibition in regimen 1 and 90% inhibition in regimen II). T/C for regimen I is 0.03 and 0.1 for regimen II. The results show ATT-11E to be a potent agent for tumor growth inhibition by two different dose regimens.

### Example 12

### This Example Demonstrates Dose Response Effect of ATT-11E on Melanoma Tumors In Vivo

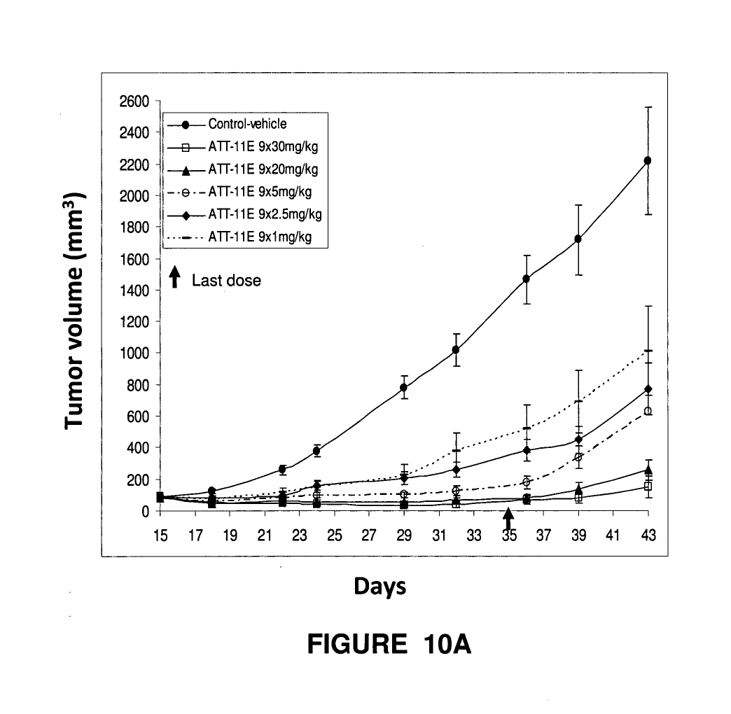

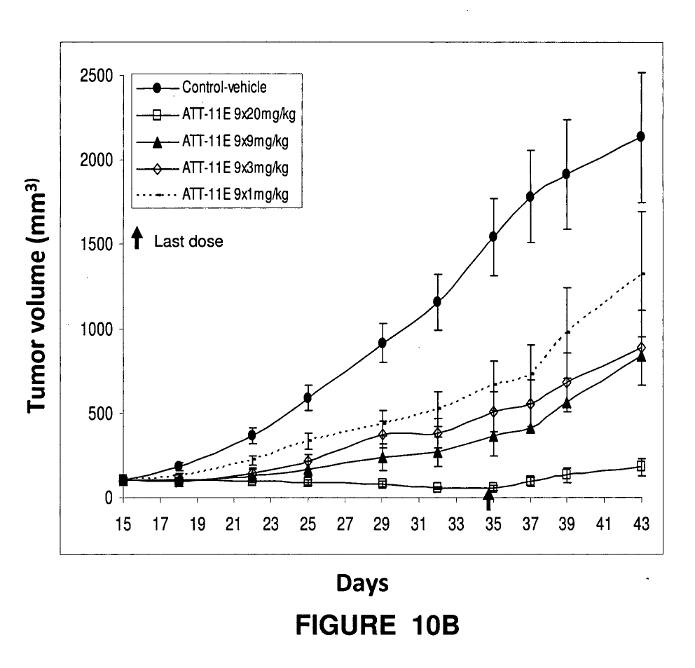

In a first experiment, increasing doses of ATT-11E were administered to mice on days 15, 17, 21, 23, 25, 28, 30, 32 and 35 post tumor cell inoculation (procedures as described above). The results of this experiment are shown in FIG. 10A as tumor volume (mm³) per day for the different doses. FIG. 10A displays a graph showing the change in tumor volume per each drug dose compared to the control.

Increasing inhibition of tumor growth and decreasing T/C values are shown for ATT-11E, as summarized in the table below:

| | | % |
|---|---|---|
| Dose | T/C | inhibition |
| 9 × 30 mg/kg | 0.07 | 93% |
| 9 × 20 mg/kg | 0.12 | 88% |
| 9 × 5 mg/kg | 0.28 | 72% |
| 9 × 2.5 mg/kg | 0.35 | 65% |
| 9 × 1 mg/kg | 0.46 | 54% |

A second experiment showing the dose response effect of ATT-11E on melanoma tumors was performed. Increasing doses of ATT-11E were administered on days 15, 17, 21, 23, 25, 28, 30, 32 and 35 post tumor cell inoculation (procedures as described above).

The results of the second experiment are shown in FIG. 10B as tumor volume (mm³) per day for the different doses. FIG. 10B displays a graph showing the change in tumor volume per each drug dose compared to the control. An arrow marks the last day of treatment.

A clear inhibitory effect on tumor growth is demonstrated for all doses of ATT-11E, as can be seen in FIG. 10B. Decreasing T/C values were shown for ATT-11E in a dose dependent manner, as summarized in the table below:

| Dose | T/C |
|---|---|
| 9 × 20 mg/kg | 0.08 |
| 9 × 9 mg/kg | 0.39 |
| 9 × 3 mg/kg | 0.41 |
| 9 × 1 mg/kg | 0.62 |

As shown in these, two experiments, ATT-11E was found to be a potent tumor growth inhibiting agent, showing a clear dose response.

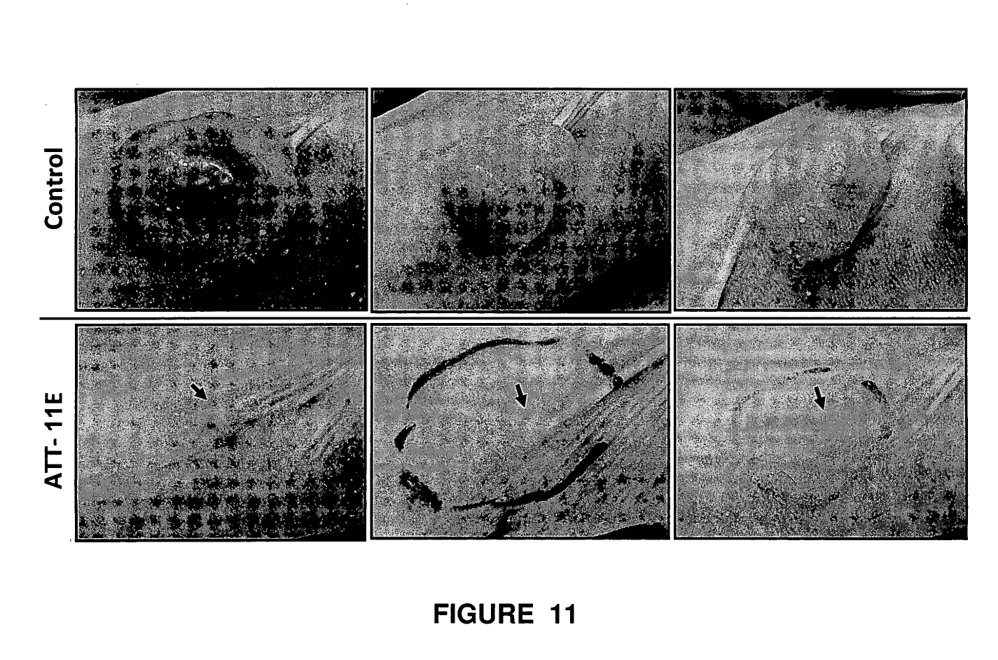

FIG. 11 (upper panel) demonstrates three representative tumors from control untreated mice 24 hours after the last administration of the vehicle. The lower panel demonstrates the tumor inoculation site arrows) 24 hours after the last administration of the drug in three representative mice treated with 9 doses of 30 mg/kg ATT-11E 3 times a week for 3 weeks.

As shown in FIG. 11, ATT-11E caused tumor regression and tumor growth inhibition, thus demonstrating its potent anti-cancer activity in vivo.

### Example 13

### This Example Evaluates Potential Adverse Effects on Body Weight of ATT-11E in tumor-Bearing Nude Mice

For the evaluation of potential adverse effects of ATT-11E, nude mice, bearing the A375 melanoma tumors and treated with ATT-11E as described above, were monitored for adverse effects immediately after drug administration, and twice a week, through one day after last dose. The evaluation protocol consisted of gross observation, registration of abnormal signs, and determination of body weight. Potential body weight loss (BWL) was calculated as % BWL=100−(BW_{dayx}/BW_{day1}×100), where day 1 is the first day of treatment and day x is the day of assessment thereafter.

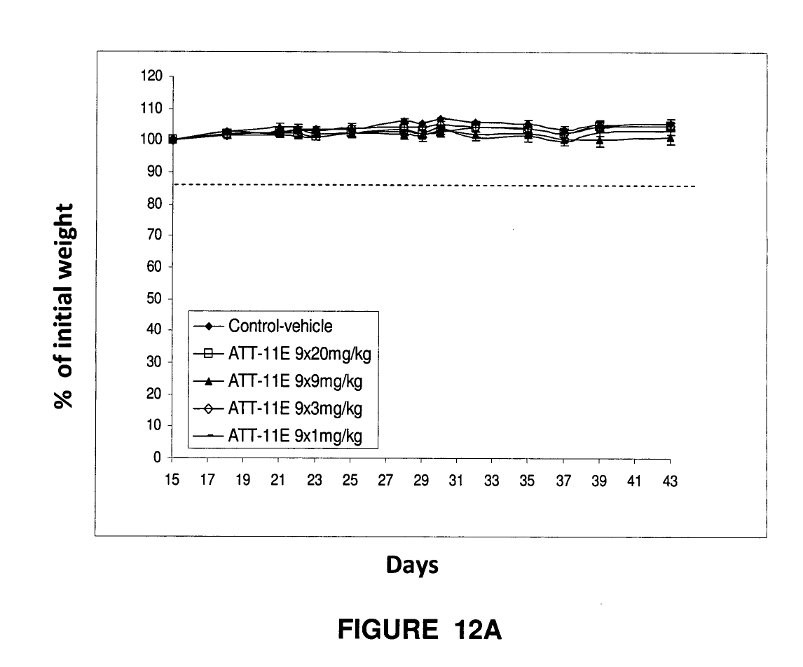

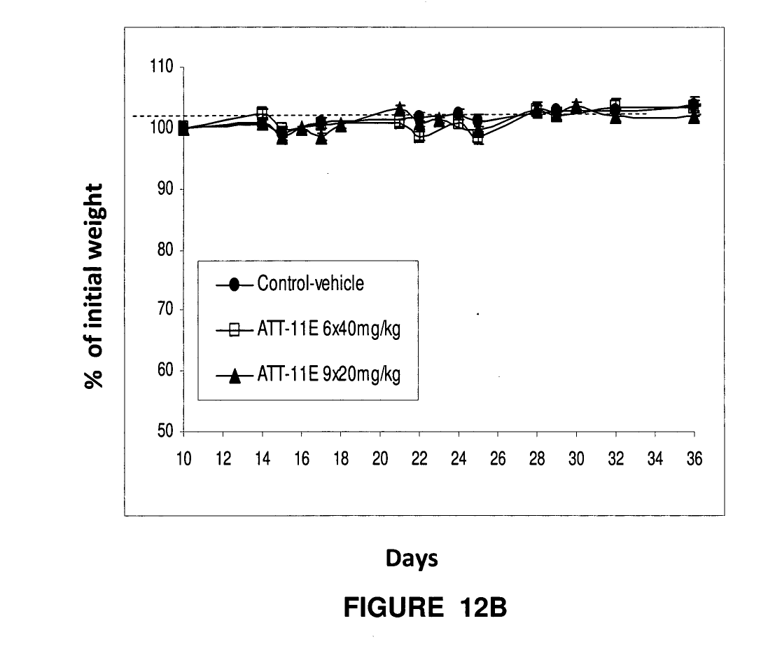

In a first experiment the following dosing regimens were tested: 9 doses of 1, 3, 9, and 20 mg/kg, and BWL of the animals was monitored. The results of this experiment are displayed in FIG. 12A in terms of % of initial weight per day starting at day 1 of treatment (day 1 of treatment is day 15 after inoculation of tumor). It can be seen that there is no discernable effect of ATT-11E on the body weight of treated animals, in all doses, compared to an untreated animal. The results show that increasing doses of ATT-11E had no adverse effect on the body weight of treated animals.

In a second experiment the effect of ATT-11E on body weight of tumor-bearing animal was tested. BWL was assessed in two different dosing regimens (6 doses of 40 mg/kg and 9 doses of 20 mg/kg). The results of the second experiment are shown in FIG. 12B. No effect of ATT-11E on body weight was demonstrated.

These two experiments demonstrate that there was no body weight loss due to the use of ATT-11E as compared to the control.

### Example 14

### In this Example, the Maximal Tolerated Dose of ATT-11E in Mice was Tested

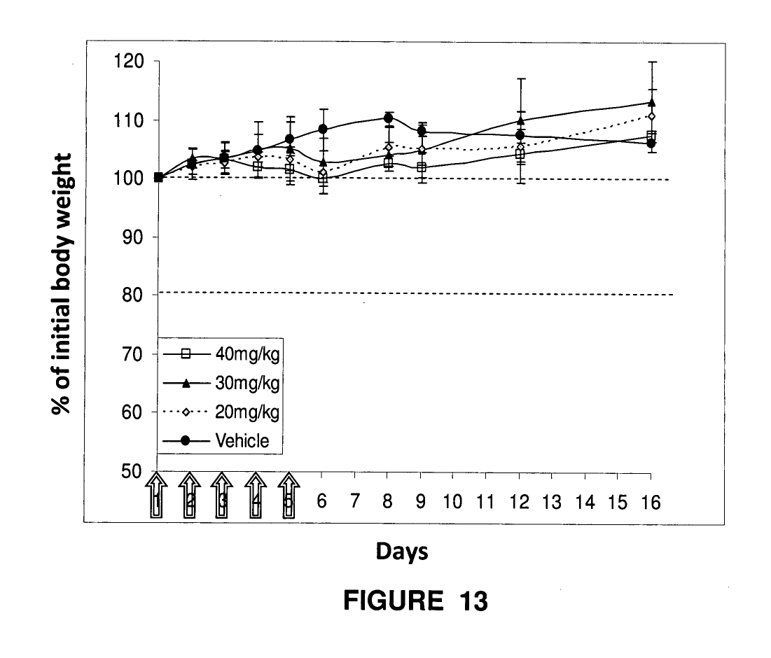

Increasing doses of ATT-11E were tested on nude mice for their effect on body weight. Healthy female athymic (nu/nu) mice (6-8 weeks) (Harlan Inc., Jerusalem, Israel) were used for determination of maximum tolerable dose (MTD). MTD was defined as the maximum dose that caused no drug-related lethality and that produced animal body weight loss of <20% of original weight. ATT-11E was dissolved in phosphate buffer (pH 7.4) containing 10% DMSO and 30% cremophor and administered by i.v. injection. ATT-11E was administered on 5 consecutive days. Body weight of mice was monitored daily and MTD was determined 24 hr post last dose. The results, graphically displayed in FIG. 13, show the effect of ATT-11E administered on 5 consecutive days (arrows mark the days of administration) in doses up to 40 mg/kg on animal body weight (% of initial weight). It can be seen that in this experiment MTD of ATT-11E was not reached, i.e., the highest dose does not affect survival and does not cause body weight loss, indicating that MTD of ATT-11E is greater than 40 mg/kg. Thus, ATT-11E does not affect animal body weight at doses up to 40 mg/kg.

### Example 15

### This Example Demonstrates the Efficacy of ATT-11TBE as an Anti-Cancer Drug In Vitro

The efficacy of ATT-11TBE as an anti-cancer drug is exemplified in cultured A375-melanoma cells in vitro, in comparison with the commercially available anti-cancer drug CPT-11 (Irinotecan), which is a pro-drug of SN-38, an anti-cancer agent, active as an inhibitor of the enzyme topoisomerase I. ATT-11TBE (Aptuit-Laurus (India) was prepared in a vehicle comprising 10% DMSO and 10% bovine serum albumin (BSA) in 0.1M sodium phosphate buffer (pH 5.8).

Melanoma (A375) cells were grown as described above. ATT-11TBE and CPT-11 were applied to the cells in increasing concentrations. The in vitro cytotoxicity was determined using a cell proliferation tetrazolium dye reduction assay, as described above.

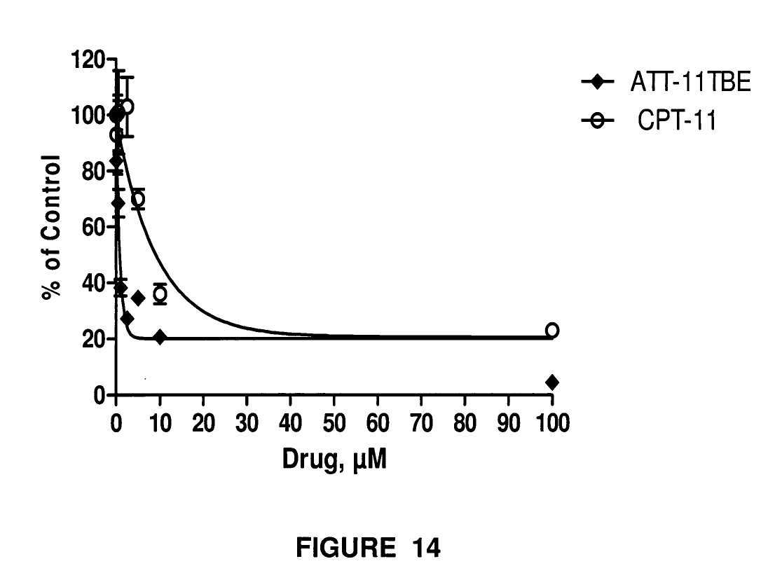

The results, displayed in FIG. 14, show the cell killing effect of ATT-11TBE on melanoma cells as a percentage of viable cells as compared to cells treated with CPT-11. The IC₅₀ value of ATT-11TBE (calculated as described above) was 0.8 μM whereas CPT-11 exhibited an IC₅₀ of 9 μM. These results indicate a higher cell killing effect of ATT-11TBE compared to CPT-11.

Thus, the cytotoxic performance of ATT-11TBE in vitro is shown to be better than that of CPT-11, indicating ATT-11 TBE as a potent cytotoxic agent.

### Example 16

### This Example Demonstrates the Efficacy of ATT-11TBE in Inducing Tumor Regression and on Inhibiting Tumor Growth in a Xenograft Model of Melanoma in Nude Mice In Vivo

Subcutaneous A375 melanoma tumors were established in mice by injection of A375 melanoma cells (0.75×10⁶ per animal per site) into the right flank region of athymic nude mice (female, 8-9 weeks, 10 animals per group). Tumor dimensions were measured with a caliper twice each week and tumor volume (mm³) was calculated using the Formula: TV=0.52 L×W², where L and W are the major and minor dimensions, respectively. Doses of 5 mg/kg and 20 mg/kg ATT-11TBE were administered to the mice (10 mice/group) 3 times a week (on days 10, 12, 14, 16, 18, 21, 23, 25 and 28 post tumor cells inoculation). Control mice were treated with the vehicle solution. The efficacy of the drug treatment assessed as inhibition of tumor growth (TVI (%)) in treated versus control tumors was calculated according to the equation: TVI(%)=(1-TIC)×100, where T and C were the tumor volume of the treated and control groups, respectively.

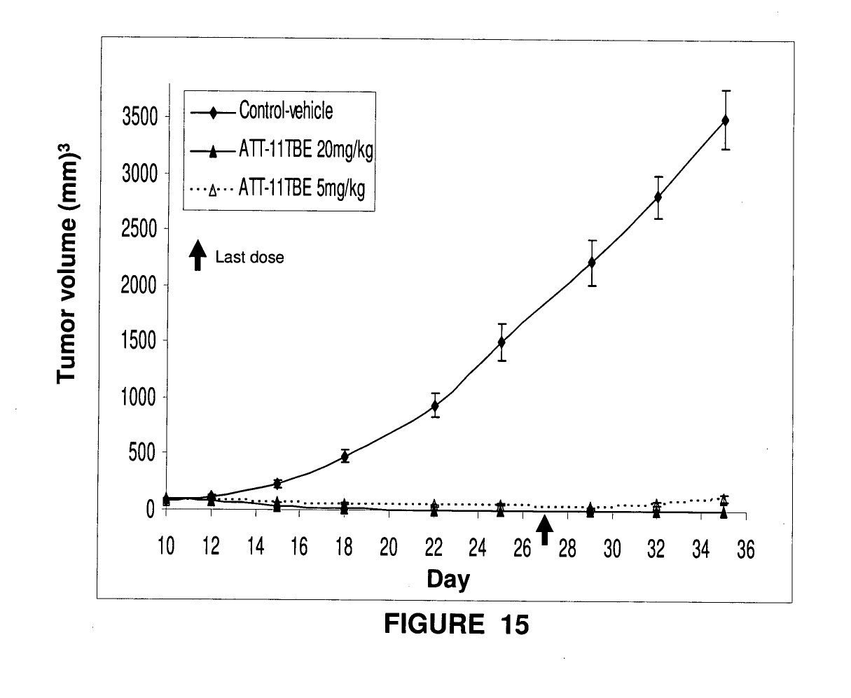

The results displayed in FIG. 15 depict tumor volume (mm³) per day of treatment. An arrow marks the last day of treatment. A dramatic inhibition of tumor volume was exhibited (97% tumor volume growth inhibition with 5 mg/kg ATT-11TBE and 100% inhibition with 20 mg/kg). These results show ATT-11TBE to be a potent anti-cancer agent for tumor growth inhibition.

### Example 17

### This Example Demonstrates the Efficacy of ATT-11TBE in Inducing Delay in Tumor Growth in a Xenograft Model of Melanoma in Nude Mice In Vivo

The rate of tumor growth following the last dose of chemotherapy was assessed for ATT-11TBE, in comparison with that of CPT-11 (Irinotecan). Drugs, i.e., ATT-11TBE or CPT-11 were administered intravenously into the tail vein of A375 melanoma-bearing mice, as described above (10 animals per group). CPT-11 was administered as hydrochloride trihydrate (Pfizer, USA), diluted from a concentrate stock solution of 100 mg/5 ml. ATT-11TBE (Aptuit-Luarus, India) was administered in a vehicle as described above. Tumor-bearing mice treated only with the vehicle solution served as controls. ATT-11TBE was administered at a dose of 20 mg/kg, given 3 times a week (on days 10, 12, 14, 16, 18, 21, 23, 25 and 28 post tumor cells inoculation). CPT-11 was administered at a dose of 75 mg/kg, given once a week for three weeks. Drug-induced delay in tumor growth was assessed as described above.

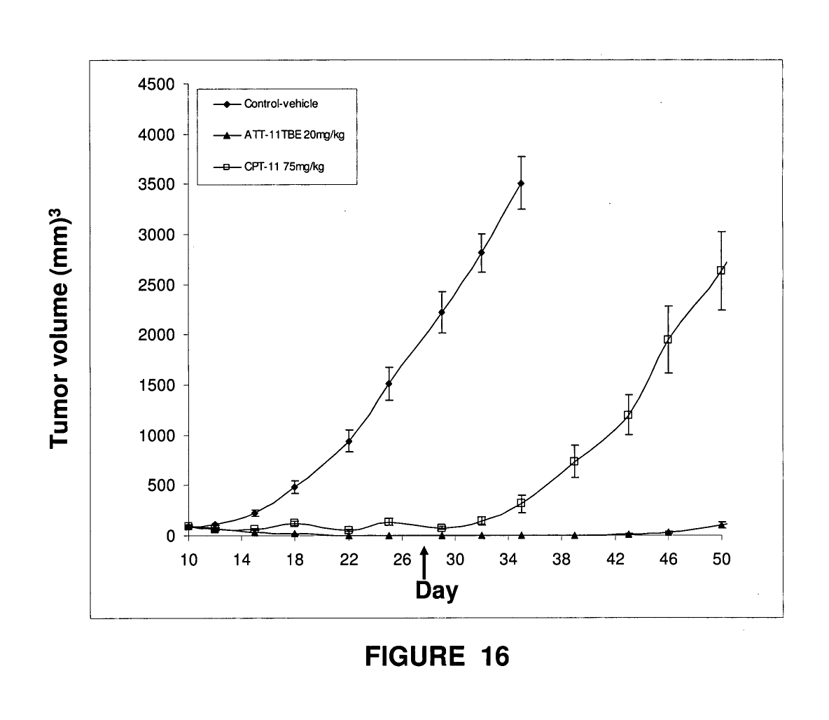

As shown in FIG. 16, ATT-11TBE (at a dose of 20 mg/kg) caused a substantial inhibition of tumor growth, with complete tumor regression, 22 days after the last dose of the drug. While CPT-11 manifested marked tumor inhibition, it never reached the complete tumor regression observed with ATT-11TBE.

Taken together, these results indicate that ATT-11TBE has potent anti-tumor properties, being able to induce both tumor regression and prolonged inhibition of tumor growth.

### Example 18

### This Example Evaluates Potential Adverse Effects of ATT-11TBE in Tumor-Bearing Nude Mice

For the evaluation of potential adverse effects of ATT-11TBE, nude mice, bearing the A375 melanoma tumors and treated with ATT-11TBE (5 mg/kg and 20 mg/kg) as described above, were monitored for adverse effects immediacy after drug administration, and also twice a week, through one day after last dose. The evaluation protocol consisted of gross observation, registration of abnormal signs, and determination of body weight. Potential body weight loss (BWL) was calculated as % BWL=100−(BW_{dayx}/BW_{day1}×100), where day 1 is the first day of treatment and day x is the day of assessment thereafter.

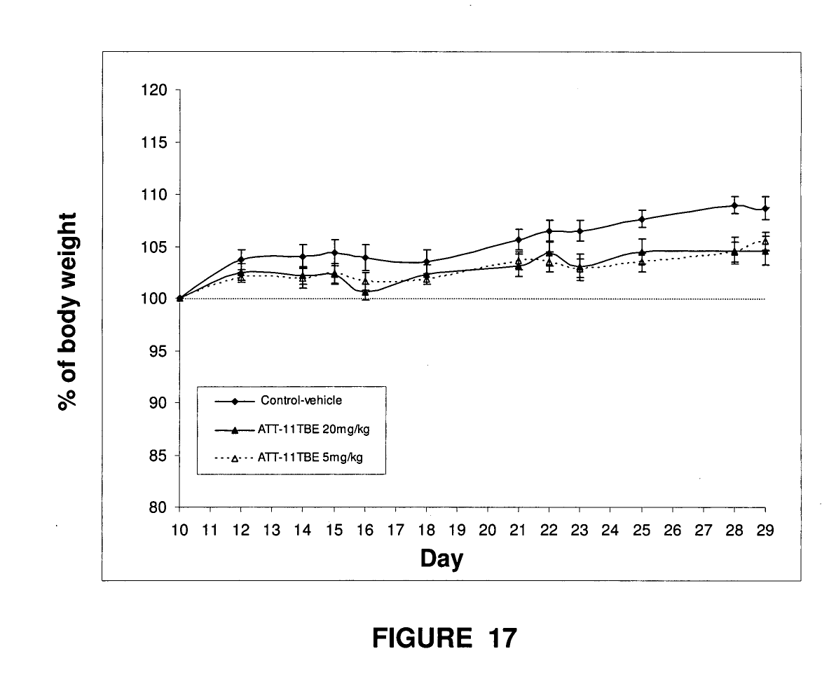

As shown in FIG. 17 in terms of % of initial weight per day starting at day 1 of treatment (day 10 after inoculation) up to day 29 of inoculation, neither loss of body weight nor other adverse effects were observed. The ATT-11TBE treated animals gained weight, with the animals treated with 5 mg/kg gaining +6% of initial body weight, and the animals treated with 20 mg/kg gaining +5%

Therefore, as assessed in these experimental systems, concurrently with its marked effect in induction of tumor regression and in inhibiting tumor growth as described above, ATT-11TBE was well tolerated at both doses, without observable adverse effects or weight loss.

### Experiment 19

### This Example Demonstrates Pharmacokinetic Properties of ATT-11TBE in Dogs

A pharmacokinetics study was performed in beagle dogs, following a single intravenous administration of ATT-11TBE, while CPT-11 served as comparator. Both drugs were administered to female beagle dogs (Auricoop Ltd, Hungary) at a dose of 6 mg/kg, with subsequent measurement of plasma concentrations over time of both the parent drugs (ATT-11 TBE or CPT-11) and the common active cytotoxic metabolite SN-38.

For this experiment, ATT-11TBE (Aptuit-Laurus, India), was dissolved in DMSO to prepare a clear stock solution of 50 mg/ml. The stock solution was diluted 10-fold in 10% bovine serum albumin solution in 0.1M sodium phosphate buffer (pH 5.8) and mixed by vortex until a homogeneous emulsion was obtained. CPT-11 (Irinotecan) hydrochloride trihydrate, Pfizer, USA) was diluted from a concentrate stock solution of 100 mg/5 ml. Fresh formulations of both drugs were prepared prior to each administration, and the drugs were administrated by a slow bolus injection within approximately 3 minutes, at a dose of 6 mg/kg (1.2 ml/kg), adjusted to the individual animal body weight.

For determination of plasma levels of ATT-11TBE, CPT-11 and SN-38, samples of approximately 3 ml of blood each were collected into EDTA coated vials, containing 75 μl dichlorvos (esterase inhibitor) solution (1.2% (V/V) dichlorvos in saline). Samples were collected once before dosing (0 min) and then at 5 min, 15 min, 30 min, 1 h, 2 h, 4 h, 8 h, 24 h, 48 h, 72 h and 96 h after dosing. Plasma was separated by centrifugation, and stored at −70° C. until HPLC analysis. WinNonlin™ software was used for the analysis of the pharmacokinetic data.

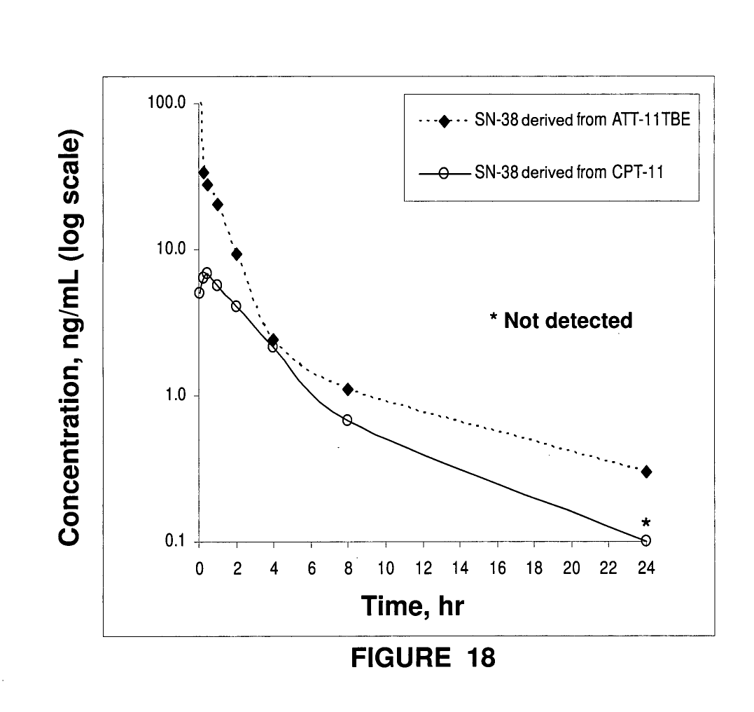

FIG. 18 shows the plasma concentrations of the common active cytotoxic metabolite SN-38 derived from ATT-11TBE or from CPT-11 vs. time. As shown, SN-38 derived from ATT-11TBE was characterized by sustained plasma levels and slow clearance as compared to CPT-11. While SN-38 derived from CPT-11 was below detection level already at 24 hours after administration, significant levels of SN-38 derived from ATT-11TBE were still detectable in the plasma at 24 hours after administration.

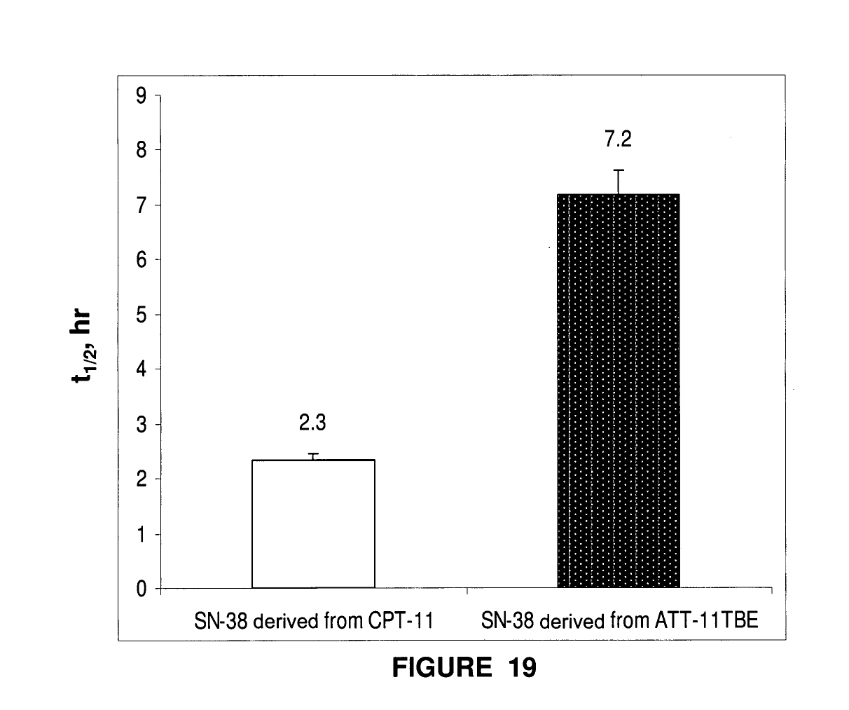

FIG. 19 shows the plasma half-life (t_{1/2}; the time taken to reach a 50% reduction in plasma drug levels) of the active common cytotoxic metabolite SN-38, derived respectively from ATT-11TBE or CPT-11, following intravenous administration to dogs. SN-38 derived from ATT-11TBE manifested a 3.1-fold longer plasma half-life, as compared to SN-38 derived from CPT-11.

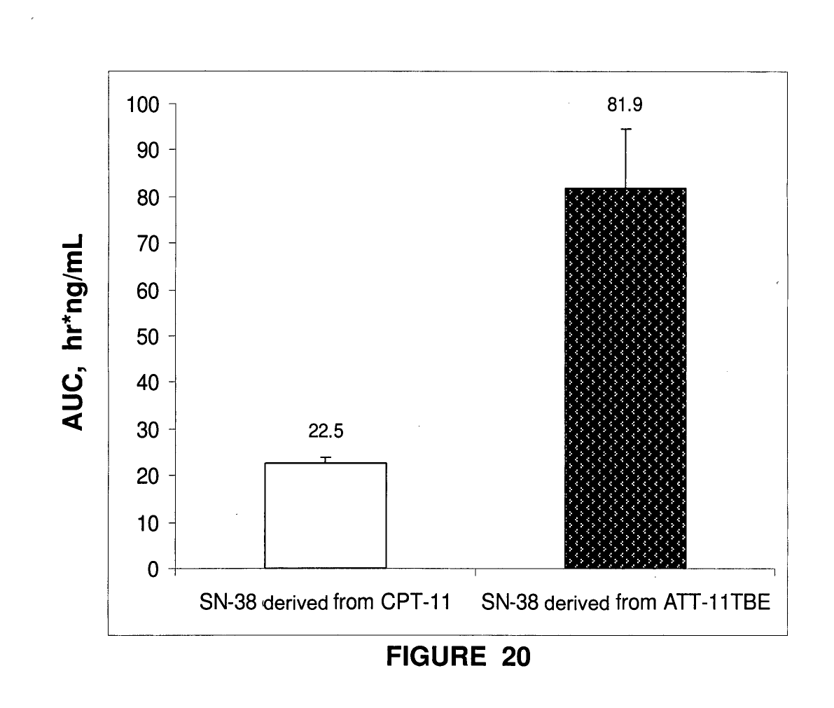

FIG. 20 shows the area under the plasma concentration/time curve (AUC) of the active cytotoxic metabolite SN-38 derived respectively from ATT-11TBE or CPT-11, following intravenous administration to beagle dogs. AUC of SN-38 derived from ATT-11TBE was 3.6-fold larger than the AUC of SN-38 derived from CPT-11.

Taken together, the results of the pharmacokinetic study in the dog demonstrate a favorable profile, with prolonged exposure of the active metabolite SN-38, as compared to CPT-11.

The above Examples demonstrate compounds and conjugates according to four embodiments of the invention. The demonstrated compounds all conform to Formula I (i.e., include D, L and Z groups as defined in Formula I). More specifically, the demonstrated compounds confer to Formula III, while demonstrating various D, L and Z groups. As shown in the Examples, compounds according to embodiments of the invention enable potent cytotoxic activity, both in vitro and in vivo, as well as potential enhanced safety profile, compared to known cytotoxic agents. Therefore, these compounds and conjugates can be beneficial in the preparation of pharmaceutical compositions, and in the treatment of cancer and immune disorders.

1. A compound represented by the structure as set forth in Formula III 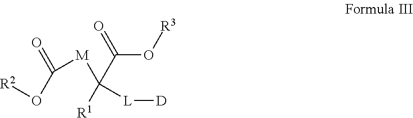
wherein R¹, R², R³ are each independently selected from hydrogen, C₁, C₂, C₃, C₄, C₅, C₆, C₇, C₈ linear or branched alkyl, or aryl or heteroaryl of one or two rings;
M is either absent, or C₁, C₂, C₃, or C₄ alkylene;
D is a cytotoxic agent; and
L is either absent, or is a linker comprising C₁, C₂, C₃, C₄, C₅, C₆, C₇, C₈ linear or branched, substituted or unsubstituted alkylene, aryl or heteroaryl, of one or two rings, carbamate, amide or ester groups, or combinations thereof; and salts, hydrates and solvates of said compound.

2. The compound according to claim 1 wherein D is selected from camptothecin, camptothecin analogue or nitrosurea.

3. The compound according to claim 2 wherein R¹, R² and R³ are each independently selected from hydrogen or C₁, C₂, C₃, C₄, C₅, C₆ linear or branched alkyl;
M is absent; and
L is either absent, or is a linker comprising C₁, C₂, C₃, C₄, C₅, C₆, C₇, C₈ linear or branched, substituted or unsubstituted alkylene, aryl or heteroaryl, of one or two rings, carbamate, amide or ester groups, or combinations thereof.

4. The compound according to claim 3, represented by the structure as set forth in Formula IV: 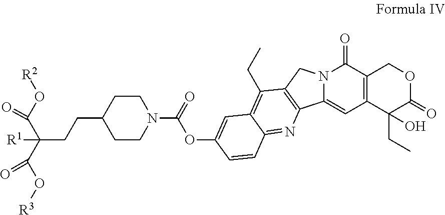
wherein R¹, R² and R³ are each independently selected from hydrogen, C₁, C₂, C₃, C₄, C₅, C₆ linear or branched alkyl.

5. The compound according to claim 4, represented by the structure as set forth in Formula VI:

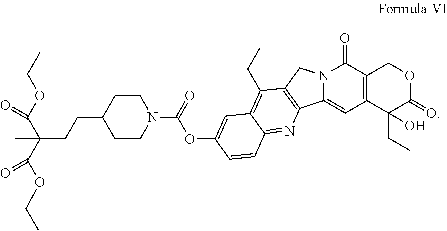

6. The compound according to claim 4, represented by the structure as set forth in Formula VI′:

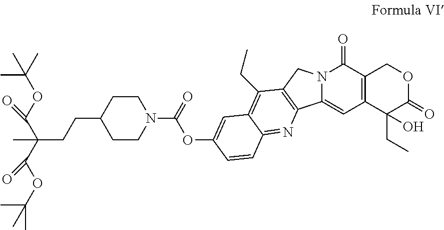

7. The compound according to claim 3, represented by the structure as set forth in Formula V:

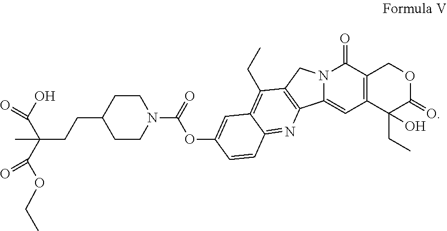

8. A compound comprising the structure as set forth in Formula II 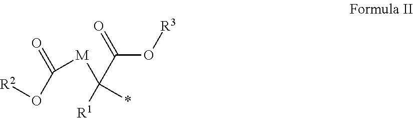
wherein R¹, R², R³ are each independently selected from hydrogen, C₁, C₂, C₃, C₄, C₅, C₆, C₇, C₈ linear or branched alkyl, aryl or heteroaryl, of one or two rings, or combinations thereof;
M is either absent or a C₁, C₂, C₃, or C₄ alkylene; and
* indicates a linkage point to a cytotoxic agent, either directly or through a linker.

9. The compound according to claim 8 wherein M is absent.

10. The compound according to claim 9 wherein R¹ is methyl.

11. The compound according to claim 8 wherein the cytotoxic agent is selected from camptothecin, camptothecin analogue, or nitrosurea.

12. The compound according to claim 8 wherein R² and R³ are each independently selected from hydrogen, C₁, C₂, C₃, or C₄, C₅, C₆, linear or branched alkyl.

13. The compound according to claim 8 comprising a linker L, selected from C₁, C₂, C₃, or C₄, C₅, C₆, C₇, C₈ linear or branched, substituted or unsubstituted, alkylene, aryl or heteroaryl, of one or two rings, carbamate, amide or ester groups, or combinations thereof, wherein the linker L is connected to the linkage point.

14. A pharmaceutical composition, comprising the compound according to claim 1, and a pharmaceutically-acceptable salt or carrier.

15. (canceled)

16. A method for the treatment of cancer and/or immune disorders, the method comprising administering to a subject, a therapeutically-effective amount of the pharmaceutical composition according to claim 14.
